SEQUENCE LISTING (23 sequences)

SEQ ID NO: 1 - DNA, 33 bp, synthetic construct
  misc_feature 1..33: note = Description of sequence: S Gene
  source 1..33: mol_type = other DNA
  modified_base 33: mod_base = OTHER; note = Quencher: 3'Black Hole Quencher_1
  modified_base 6: mod_base = OTHER; note = Fluorophores Modification: iFluorT dye
  modified_base 1: mod_base = OTHER; note = locked nucleic acid (LNA) nucleotide is Guanine
  modified_base 5: mod_base = OTHER; note = locked nucleic acid (LNA) nucleotide is Cytosine
  modified_base 7: mod_base = OTHER; note = locked nucleic acid (LNA) nucleotide is Guanine
  modified_base 10: mod_base = OTHER; note = locked nucleic acid (LNA) nucleotide is Adenine
  modified_base 13: mod_base = OTHER; note = locked nucleic acid (LNA) nucleotide is Cytosine
  modified_base 16: mod_base = OTHER; note = locked nucleic acid (LNA) nucleotide is Guanine
gcttctgctaatcttgctgctagcaagattagc

SEQ ID NO: 2 - DNA, 33 bp, synthetic construct
  misc_feature 1..33: note = Description of sequence: S Gene
  source 1..33: mol_type = other DNA
  modified_base 33: mod_base = OTHER; note = Quencher: 3'Black Hole Quencher_1
  modified_base 10: mod_base = OTHER; note = Fluorophores Modification: iFluorT dye
  modified_base 1: mod_base = OTHER; note = locked nucleic acid (LNA) nucleotide is Guanine
  modified_base 4: mod_base = OTHER; note = locked nucleic acid (LNA) nucleotide is Adenine
  modified_base 7: mod_base = OTHER; note = locked nucleic acid (LNA) nucleotide is Cytosine
  modified_base 9: mod_base = OTHER; note = locked nucleic acid (LNA) nucleotide is Guanine
  modified_base 12: mod_base = OTHER; note = locked nucleic acid (LNA) nucleotide is Guanine
  modified_base 15: mod_base = OTHER; note = locked nucleic acid (LNA) nucleotide is Guanine
gcaacacagttgctgattctcaatcagcaactg

SEQ ID NO: 3 - DNA, 34 bp, synthetic construct
  misc_feature 1..34: note = Description of sequence: N Gene
  source 1..34: mol_type = other DNA
  modified_base 1: mod_base = OTHER; note = locked nucleic acid (LNA) nucleotide is Cytosine
  modified_base 4: mod_base = OTHER; note = locked nucleic acid (LNA) nucleotide is Guanine
  modified_base 7: mod_base = OTHER; note = locked nucleic acid (LNA) nucleotide is Cytosine
  modified_base 10: mod_base = OTHER; note = locked nucleic acid (LNA) nucleotide is Adenine
  modified_base 13: mod_base = OTHER; note = locked nucleic acid (LNA) nucleotide is Thymine
  modified_base 16: mod_base = OTHER; note = locked nucleic acid (LNA) nucleotide is Thymine
cgggtgccaatgtgatcttttgagatcacattgg

SEQ ID NO: 4 - DNA, 32 bp, synthetic construct
  misc_feature 1..32: note = Description of sequence: N Gene
  source 1..32: mol_type = other DNA
  modified_base 1: mod_base = OTHER; note = locked nucleic acid (LNA) nucleotide is Cytosine
  modified_base 4: mod_base = OTHER; note = locked nucleic acid (LNA) nucleotide is Thymine
  modified_base 7: mod_base = OTHER; note = locked nucleic acid (LNA) nucleotide is Cytosine
  modified_base 10: mod_base = OTHER; note = locked nucleic acid (LNA) nucleotide is Guanine
  modified_base 13: mod_base = OTHER; note = locked nucleic acid (LNA) nucleotide is Adenine
  modified_base 16: mod_base = OTHER; note = locked nucleic acid (LNA) nucleotide is Cytosine
ccattgccagccattctagctagaatggctgg

SEQ ID NO: 5 - DNA, 30 bp, synthetic construct
  misc_feature 1..30: note = Description of sequence: N Gene
  source 1..30: mol_type = other DNA
  modified_base 1: mod_base = OTHER; note = locked nucleic acid (LNA) nucleotide is Guanine
  modified_base 4: mod_base = OTHER; note = locked nucleic acid (LNA) nucleotide is Guanine
  modified_base 7: mod_base = OTHER; note = locked nucleic acid (LNA) nucleotide is Thymine
  modified_base 10: mod_base = OTHER; note = locked nucleic acid (LNA) nucleotide is Guanine
  modified_base 13: mod_base = OTHER; note = locked nucleic acid (LNA) nucleotide is Cytosine
  modified_base 16: mod_base = OTHER; note = locked nucleic acid (LNA) nucleotide is Thymine
gttgagtgagagcggtgaacaccgctctca

SEQ ID NO: 6 - DNA, 31 bp, synthetic construct
  misc_feature 1..31: note = Description of sequence: Delta
  source 1..31: mol_type = other DNA
  modified_base 1: mod_base = OTHER; note = locked nucleic acid (LNA) nucleotide is Cytosine
  modified_base 4: mod_base = OTHER; note = locked nucleic acid (LNA) nucleotide is Cytosine
  modified_base 7: mod_base = OTHER; note = locked nucleic acid (LNA) nucleotide is Thymine
  modified_base 9: mod_base = OTHER; note = locked nucleic acid (LNA) nucleotide is Thymine
  modified_base 11: mod_base = OTHER; note = locked nucleic acid (LNA) nucleotide is Cytosine
  modified_base 14: mod_base = OTHER; note = locked nucleic acid (LNA) nucleotide is Cytosine
  modified_base 16: mod_base = OTHER; note = locked nucleic acid (LNA) nucleotide is Adenine
cctcactttccatccaacttttggatggaaa

SEQ ID NO: 7 - DNA, 36 bp, synthetic construct
  misc_feature 1..36: note = Description of sequence: Delta
  source 1..36: mol_type = other DNA
  modified_base 1: mod_base = OTHER; note = locked nucleic acid (LNA) nucleotide is Cytosine
  modified_base 4: mod_base = OTHER; note = locked nucleic acid (LNA) nucleotide is Cytosine
  modified_base 7: mod_base = OTHER; note = locked nucleic acid (LNA) nucleotide is Cytosine
  modified_base 10: mod_base = OTHER; note = locked nucleic acid (LNA) nucleotide is Guanine
  modified_base 13: mod_base = OTHER; note = locked nucleic acid (LNA) nucleotide is Thymine
  modified_base 16: mod_base = OTHER; note = locked nucleic acid (LNA) nucleotide is Guanine
cgccgacgagaattagtctgagtcgactaattctcg

SEQ ID NO: 8 - DNA, 30 bp, synthetic construct
  misc_feature 1..30: note = Description of sequence: Gene- N2 (NC_045512.2) Location 29190-29210
  source 1..30: mol_type = other DNA
  modified_base 30: mod_base = OTHER; note = Quencher: 3'Black Hole Quencher_2
  modified_base 7: mod_base = OTHER; note = Fluorophores Modification: Int Cy3 Freedom Dye
  modified_base 1: mod_base = OTHER; note = locked nucleic acid (LNA) nucleotide is Cytosine
  modified_base 4: mod_base = OTHER; note = locked nucleic acid (LNA) nucleotide is Adenine
  modified_base 7: mod_base = OTHER; note = locked nucleic acid (LNA) nucleotide is Cytosine
  modified_base 10: mod_base = OTHER; note = locked nucleic acid (LNA) nucleotide is Thymine
  modified_base 13: mod_base = OTHER; note = locked nucleic acid (LNA) nucleotide is Guanine
  modified_base 16: mod_base = OTHER; note = locked nucleic acid (LNA) nucleotide is Cytosine
ctgaagcgctgggggcaaattcccccagcg

SEQ ID NO: 9 - DNA, 34 bp, synthetic construct; identical to SEQ ID NO: 3
  misc_feature 1..34: note = Description of sequence: Gene-N4 (NC_045512.2) Location 28699-28719
  source 1..34: mol_type = other DNA
  modified_base 34: mod_base = OTHER; note = Quencher: 3'Black Hole Quencher_2
  modified_base 7: mod_base = OTHER; note = Fluorophores Modification: Int Cy3 Freedom Dye
  modified_base 1: mod_base = OTHER; note = locked nucleic acid (LNA) nucleotide is Cytosine
  modified_base 4: mod_base = OTHER; note = locked nucleic acid (LNA) nucleotide is Guanine
  modified_base 7: mod_base = OTHER; note = locked nucleic acid (LNA) nucleotide is Cytosine
  modified_base 10: mod_base = OTHER; note = locked nucleic acid (LNA) nucleotide is Adenine
  modified_base 13: mod_base = OTHER; note = locked nucleic acid (LNA) nucleotide is Thymine
  modified_base 16: mod_base = OTHER; note = locked nucleic acid (LNA) nucleotide is Thymine

SEQ ID NO: 10 - DNA, 32 bp, synthetic construct
  misc_feature 1..32: note = Description of sequence: Gene- N7 (NC_045512.2) Location 28531 28551
  source 1..32: mol_type = other DNA
  modified_base 32: mod_base = OTHER; note = Quencher: 3'Black Hole Quencher_2
  modified_base 7: mod_base = OTHER; note = Fluorophores Modification: Int Cy3 Freedom Dye
  modified_base 1: mod_base = OTHER; note = locked nucleic acid (LNA) nucleotide is Cytosine
  modified_base 4: mod_base = OTHER; note = locked nucleic acid (LNA) nucleotide is Cytosine
  modified_base 7: mod_base = OTHER; note = locked nucleic acid (LNA) nucleotide is Guanine
  modified_base 10: mod_base = OTHER; note = locked nucleic acid (LNA) nucleotide is Guanine
  modified_base 13: mod_base = OTHER; note = locked nucleic acid (LNA) nucleotide is Cytosine
  modified_base 16: mod_base = OTHER; note = locked nucleic acid (LNA) nucleotide is Cytosine
cgtctggtagctcttcggtagcgaagagctac

SEQ ID NO: 11 - DNA, 31 bp, synthetic construct; identical to SEQ ID NO: 6
  misc_feature 1..31: note = Description of sequence: Gene- delta F157 (B.1 .617.2) (OX014251.1) Location 22011 22028
  source 1..31: mol_type = other DNA
  modified_base 31: mod_base = OTHER; note = Quencher: 3'Black Hole Quencher_2
  modified_base 1: mod_base = OTHER; note = Fluorophores Modification: Int Cy3 Freedom Dye
  modified_base 1: mod_base = OTHER; note = locked nucleic acid (LNA) nucleotide is Cytosine
  modified_base 4: mod_base = OTHER; note = locked nucleic acid (LNA) nucleotide is Cytosine
  modified_base 7: mod_base = OTHER; note = locked nucleic acid (LNA) nucleotide is Thymine
  modified_base 9: mod_base = OTHER; note = locked nucleic acid (LNA) nucleotide is Thymine
  modified_base 11: mod_base = OTHER; note = locked nucleic acid (LNA) nucleotide is Cytosine
  modified_base 14: mod_base = OTHER; note = locked nucleic acid (LNA) nucleotide is Cytosine
  modified_base 16: mod_base = OTHER; note = locked nucleic acid (LNA) nucleotide is Adenine

SEQ ID NO: 12 - DNA, 40 bp, synthetic construct
  misc_feature 1..40: note = Description of sequence: Gene L452R (B.1 .617.2) (OX014251.1) Location 22892 22917
  source 1..40: mol_type = other DNA
  modified_base 40: mod_base = OTHER; note = Quencher: 3'Black Hole Quencher_2 conjugated to guanine
  modified_base 1: mod_base = OTHER; note = Fluorophores Modification: Int Cy3 Freedom Dye
  modified_base 1: mod_base = OTHER; note = locked nucleic acid (LNA) nucleotide is Cytosine
  modified_base 3: mod_base = OTHER; note = locked nucleic acid (LNA) nucleotide is Adenine
  modified_base 5: mod_base = OTHER; note = locked nucleic acid (LNA) nucleotide is Adenine
  modified_base 7: mod_base = OTHER; note = locked nucleic acid (LNA) nucleotide is Cytosine
  modified_base 9: mod_base = OTHER; note = locked nucleic acid (LNA) nucleotide is Guanine
  modified_base 11: mod_base = OTHER; note = locked nucleic acid (LNA) nucleotide is Adenine
  modified_base 13: mod_base = OTHER; note = locked nucleic acid (LNA) nucleotide is Thymine
  modified_base 16: mod_base = OTHER; note = locked nucleic acid (LNA) nucleotide is Thymine
ctataccggtaattataattaccaccaattataattaccg

SEQ ID NO: 13 - DNA, 33 bp, synthetic construct
  misc_feature 1..33: note = Description of sequence: Gene delta H69 (BA.1) (OR624070.1) Location 21682-21703
  source 1..33: mol_type = other DNA
  modified_base 33: mod_base = OTHER; note = Quencher: 3'Black Hole Quencher_2
  modified_base 7: mod_base = OTHER; note = Fluorophores Modification: Int Cy3 Freedom Dye
  modified_base 1: mod_base = OTHER; note = locked nucleic acid (LNA) nucleotide is Cytosine
  modified_base 4: mod_base = OTHER; note = locked nucleic acid (LNA) nucleotide is Guanine
  modified_base 7: mod_base = OTHER; note = locked nucleic acid (LNA) nucleotide is Adenine
  modified_base 10: mod_base = OTHER; note = locked nucleic acid (LNA) nucleotide is Guanine
  modified_base 13: mod_base = OTHER; note = locked nucleic acid (LNA) nucleotide is Thymine
  modified_base 16: mod_base = OTHER; note = locked nucleic acid (LNA) nucleotide is Adenine
ccagatatagcatggaaccaagttccatgctat

SEQ ID NO: 14 - DNA, 33 bp, synthetic construct
  misc_feature 1..33: note = Description of sequence: Gene Influenza A (H1N1,NP) (OR_637833.1) Location 616-637
  source 1..33: mol_type = other DNA
  modified_base 33: mod_base = OTHER; note = Quencher: 3'Black Hole Quencher_2
  modified_base 7: mod_base = OTHER; note = Fluorophores Modification: Int Cy3 Freedom Dye
  modified_base 1: mod_base = OTHER; note = locked nucleic acid (LNA) nucleotide is Guanine
  modified_base 4: mod_base = OTHER; note = locked nucleic acid (LNA) nucleotide is Cytosine
  modified_base 7: mod_base = OTHER; note = locked nucleic acid (LNA) nucleotide is Thymine
  modified_base 10: mod_base = OTHER; note = locked nucleic acid (LNA) nucleotide is Cytosine
  modified_base 13: mod_base = OTHER; note = locked nucleic acid (LNA) nucleotide is Cytosine
  modified_base 16: mod_base = OTHER; note = locked nucleic acid (LNA) nucleotide is Thymine
gtccattttcacccctccagaagaggggtgaaa

SEQ ID NO: 15 - DNA, 35 bp, synthetic construct
  misc_feature 1..35: note = Description of sequence: Gene RSV (OQ_848527.1) Location 5944- 5966
  source 1..35: mol_type = other DNA
  modified_base 35: mod_base = OTHER; note = Quencher: 3'Black Hole Quencher_2
  modified_base 7: mod_base = OTHER; note = Fluorophores Modification: Int Cy3 Freedom Dye
  modified_base 1: mod_base = OTHER; note = locked nucleic acid (LNA) nucleotide is Cytosine
  modified_base 4: mod_base = OTHER; note = locked nucleic acid (LNA) nucleotide is Guanine
  modified_base 7: mod_base = OTHER; note = locked nucleic acid (LNA) nucleotide is Cytosine
  modified_base 10: mod_base = OTHER; note = locked nucleic acid (LNA) nucleotide is Thymine
  modified_base 13: mod_base = OTHER; note = locked nucleic acid (LNA) nucleotide is Thymine
  modified_base 16: mod_base = OTHER; note = locked nucleic acid (LNA) nucleotide is Guanine
ctggctcgattgtttgttgcttgaacaaacaatcg

SEQ ID NO: 16 - DNA, 21 bp, synthetic construct
  misc_feature 1..21: note = Description of sequence: Primer
  source 1..21: mol_type = other DNA
aatttgcccccagcgcttcag

SEQ ID NO: 17 - DNA, 21 bp, synthetic construct
  misc_feature 1..21: note = Description of sequence: Primer
  source 1..21: mol_type = other DNA
aaaagatcacattggcacccg

SEQ ID NO: 18 - DNA, 21 bp, synthetic construct
  misc_feature 1..21: note = Description of sequence: Primer
  source 1..21: mol_type = other DNA
ctaccgaagagctaccagacg

SEQ ID NO: 19 - DNA, 18 bp, synthetic construct
  misc_feature 1..18: note = Description of sequence: Primer
  source 1..18: mol_type = other DNA
aaagttggatggaaagtg

SEQ ID NO: 20 - DNA, 26 bp, synthetic construct
  misc_feature 1..26: note = Description of sequence: Primer
  source 1..26: mol_type = other DNA
ggtggtaattataattaccggtatag

SEQ ID NO: 21 - DNA, 22 bp, synthetic construct
  misc_feature 1..22: note = Description of sequence: Primer
  source 1..22: mol_type = other DNA
cttggttccatgctatatctgg

SEQ ID NO: 22 - DNA, 22 bp, synthetic construct
  misc_feature 1..22: note = Description of sequence: Primer
  source 1..22: mol_type = other DNA
ttctggaggggtgaaaatggac

SEQ ID NO: 23 - DNA, 23 bp, synthetic construct
  misc_feature 1..23: note = Description of sequence: Primer
  source 1..23: mol_type = other DNA
caagcaacaaacaatcgagccag

(19) United States
(12) Patent Application Publication
(10) Pub. No.: US 2026/0210962 A1
(43) Pub. Date: Jul. 23, 2026

(54) MULTIPARAMETRIC INTEGRATED MOLECULAR DETECTION OF VIRAL ANTIGENS AND VIRAL NUCLEIC ACIDS

(71) Applicant: Ohio State Innovation Foundation, Columbus, OH (US)

(72) Inventors: Eduardo REATEGUI, Columbus, OH (US); Truc Kim NGUYEN, Columbus, OH (US)

(21) Appl. No.: 19/140,526

(22) PCT Filed: Dec. 19, 2023
(86) PCT No.: PCT/US2023/084743

§ 371 Date: Jun. 18, 2025

Related U.S. Application Data
(60) Provisional application No. 63/387,991, filed on Dec. 19, 2022.

(51) Int. Cl. G01N 33/569; C12Q 1/70; G01N 33/543

(52) U.S. Cl. CPC G01N 33/56983; C12Q 1/701; G01N 33/54373; C12Q 2600/156; G01N 2333/11; G01N 2333/135; G01N 2333/165; G01N 2469/10

(57) ABSTRACT

Disclosed herein are methods for detecting and treating viral infections. Disclosed is a method of detecting a virus, comprising obtaining a biological sample; capturing a plurality of membranous particles from the biological sample; measuring antigens and nucleic acid levels in the membranous particles or single virions from the biological sample; and measuring an amount of a viral RNA; wherein a virus is detected when the viral protein level or the amount of viral RNA is increased in comparison to a control sample.

### CROSS-REFERENCE TO RELATED APPLICATIONS

This PCT application claims priority to, and the benefit of, U.S. Provisional Patent Application No. 63/387,991, filed Dec. 19, 2022, which is incorporated by reference herein in its entirety.

### STATEMENT REGARDING FEDERALLY SPONSORED RESEARCH

This invention was made with government support under TR003807 awarded by the National Institutes of Health. The government has certain rights in the invention.

### REFERENCE TO SEQUENCE LISTING

The sequence listing submitted on Dec. 19, 2023, as an .XML file entitled “103361-406WO1_ST26” created on Dec. 18, 2023, and having a file size of 74,717 bytes is hereby incorporated by reference pursuant to 37 C.F.R. § 1.52(e)(5).

### FIELD

Disclosed herein are methods for detecting and treating viral infections.

### BACKGROUND

The emergence of infectious diseases is rising and is dominated by zoonoses, which are the transmission of pathogens from animals to humans that originate via a myriad of interspecies interactions. Human history is concomitant with zoonoses, begetting pandemics, epidemics, and endemics that have plagued the human experience, the former two requiring interpersonal transmission and the latter typically contained in the individual. Although humans have coexisted with zoonotic pathogens, their outbreaks continue to disrupt the social fabric at an individual level, such as inducing psychological distress, or at the societal level, such as burdening the economy. The coronavirus disease of 2019 (COVID-19) caused by the severe acute respiratory syndrome coronavirus 2 (SARS-CoV-2) is no exception, resulting in the reported infection of 767 million people and the deaths of 6.9 million worldwide to date. While the number of deaths is staggering and continues to increase, in the United States of America (USA), excess deaths were disproportionately higher for Black, Latino, and American Indian/Alaska Native persons, exacerbating racial inequities across the country. With global warming and human land use encouraging the interaction of species via habitat reduction, the number of zoonoses is expected to increase. Therefore, methods to slow the transmission of zoonoses via rapidly tunable diagnostic assays that provide highly sensitive detection, molecular subtyping, and follow-up monitoring of pathogens are necessary to mitigate future epidemics via containment measures.

While highly sensitive, nucleic-acid-based technologies are limited at detecting genetic mutations, novel zoonoses, or low-virion counts, whereas antigen-based technologies require post-acute immune responses. Quantitative reverse-transcriptase polymerase chain reaction (qRT-PCR), a nucleic-acid-based assay, was the primary diagnostic utilized to combat the transmission of the SARS-CoV-2 virion despite its false-negative results and requirement for laboratory equipment and reagents. On the other hand, rapid-antigen tests that target intrinsic virion proteins granted accessibility to facile COVID-19 testing for the general public but are less sensitive than qRT-PCR. Given the limitations described above, bulk-analysis diagnostics are subject to the dilution of virions within the biofluid. The compositions and methods disclosed herein address the need for a novel technology required as a response to the shortcomings of traditional diagnostics to enhance sensitivities and specificities.

### SUMMARY

Virions are biogenetically and morphologically similar to extracellular vesicles (EVs), which are cell-derived lipid nanoparticles containing bioactive molecules. EVs with their unique biomolecular signatures unravel their vast heterogeneity, which has promoted the engineering of in situ single-EV technologies. Coupling in situ labeling with high-resolution microscopy, such as total internal reflection fluorescence microscopy (TIRFM), has further enabled the colocalization of proteins and nucleic acids in single EVs. TIRFM is utilized to investigate single-virion dynamics upon entry or release from the plasma membrane. Therefore, the translation of single-EV technologies to single virions can be used to screen for both the presence of virions and infected tissue. Combining TIRFM and single-EV labeling techniques provides a unique perspective into single-virion biomolecular signatures via colocalization of antigenic and nucleic acid detection.

Disclosed herein is a Biochip Antigen and RNA Assay (BARA), which isolates single virions and virion-infected host-derived EVs (IHD-EVs) from complex biofluids via positive immunoselection and infection mechanisms. The BARA combines immunofluorescence (IF) and fluorescent in situ hybridization (FISH) with TIRFM providing high-resolution qualitative and quantitative antigenic and nucleic acid expression of single particles. The BARA is validated with the SARS-CoV-2 virion following the guidelines for Emergency Use Authorization (EUA) regulated by the United States Food and Drug Administration (FDA). By progressing toward single-virion detection, the BARA outperformed quantitative reverse transcription polymerase chain reaction (qRT-PCR) by one order of magnitude regarding the limit of detection (LoD), which upon combining antigenic and nucleic acid detection, yielded sensitivities of 100% and 95% and specificities of 100% and 100% for saliva and nasopharyngeal swab (NS) samples, respectively. Furthermore, the BARA revealed the continued long-term expression of virion-RNA in IHD-EVs from post-acute sequelae of COVID-19 (PASC) patient plasma. The success of the work provides a tunable framework to interrogate single virions and long-term infections via the simultaneous detection of biomolecules in single particles, which can be easily adapted by customizing the antibodies and proteins for immunoselection and the probes for their subsequent detection.

In some aspects, disclosed herein a method of detecting a viral antigen and a viral nucleic acid in a subject, comprising:
obtaining a biological sample from the subject;
capturing a virion from the biological sample; or
capturing a membranous particle from the biological sample; and
measuring the viral antigen and the viral nucleic acid level in the biological sample;
wherein the virion or membranous particle is immobilized on a biochip.

In some embodiments, the biochip comprises of a plasmonic surface.

In some embodiments, the viral nucleic acid comprises RNA.

In some embodiments, the viral antigen comprises a viral protein.

In some embodiments, the viral antigen or viral nucleic acid is detected when the viral antigen and the viral nucleic acid levels are increased in comparison to a control sample.

In some embodiments, the virion comprises a coronavirus virion, an influenza virus virion, or a respiratory syncytial virus (RSV) virion.

In some embodiments, the membranous particle comprises a virion-infected host-derived extracellular vesicle.

In some aspects, disclosed herein a method of treating a viral infection in a subject, comprising:
obtaining a biological sample from the subject;
capturing a virion from the biological sample: or
capturing a membranous particle from the biological sample;
measuring a viral antigen or a viral nucleic acid level in the biological sample;
wherein the virion or membranous particle is immobilized on a biochip; and
administering an antiviral agent if a viral infection is detected.

In some embodiments, the biochip comprises of a plasmonic surface.

In some embodiments, the viral infection is detected when the viral antigen or nucleic acid level is increased in comparison to a control sample.

In some aspects, disclosed herein a method for high throughput multiplexing for simultaneous detection of a viral antigen and a viral nucleic acid, the method comprising the steps of:
immobilizing a virion and/or a membranous particle on a plasmonic surface;
detecting the viral antigen and the viral nucleic acid simultaneously derived from the virion and/or membranous particle;
wherein the detection method comprises fluorescence, in situ hybridization, enzyme linked immunosorbent assay, flow cytometry, or microscopy.

In some embodiments, the viral antigen and the viral nucleic acid are detected with a sensitivity of at least 80%.

In some embodiments, the viral antigen and the viral nucleic acid are detected with a specificity of at least 95%.

### BRIEF DESCRIPTION OF FIGURES

The accompanying figures, which are incorporated in and constitute a part of this specification, illustrate several aspects described below.

FIGS. 1A, 1B, 1C, 1D, 1E and IF depict single virion antigenic and nucleic acid detection with the BARA. FIG. 1A shows the BARA assembly and schematic mechanism for detecting antigens and nucleic acids utilizing immunofluorescence (IF) and fluorescent in situ hybridization (FISH), respectively. Detection is performed on single virions and infected extracellular vesicles (EVs) derived from complex biofluids, such as blood plasma, saliva, and nasopharyngeal (nasal) swabs (NS). FIG. 1B represents total internal reflection fluorescence microscopy (TIRFM) images illustrating the spike glycoprotein on single virions and nucleocapsid-encoding RNA in single virions, and IHD-EVs, accompanied by their corresponding fluorescence intensity histograms.

FIG. 1C shows transmission electron microscope (TEM) image of SARS-CoV-2 virions showcasing the visible spike glycoprotein forming coronavirus corona. FIG. 1D represents TIRFM images demonstrating SARS-CoV-2 antigen and nucleocapsid-encoding RNA on a single SARS-CoV-2 virion and the combination of detection methods, revealing colocalization of fluorescent signals in a localized domain. FIG. 1E Representative TIRFM images displaying the colocalization of antigens and nucleic acids for Influenza A. FIG. 1F represents TIRFM images showing the colocalization of antigens and nucleic acids for respiratory syncytial virus (RSV).

FIGS. 2A, 2B, 2C, 2D, 2E, 2F and 2G depict performance evaluation of the BARA. FIG. 2A shows comparative evaluation of antigenic and nucleic acid signals from the BARA in comparison to standard qRT-PCR. Inactivated SARS-CoV-2 virions were spiked in PBS at concentrations ranging from 0 to 1×10\hat{ }6 particles/well (n=3, error bars indicate the standard deviation). FIG. 2B shows detection results at the LoD of the BARA (n=20 for LoD, n=4 for pooled healthy donor saliva), demonstrating 20/20 detectable results for the antigen test and 19/20 detectable results for the RNA test. FIG. 2C shows stability analysis of the BARA with frozen saliva specimens spiked at different LoDs (n=69 with at least 10 replicates for 1-2×LoD, 3-5×LoD, and pooled healthy donor saliva conditions). FIG. 2D shows stability analysis of the BARA with fresh saliva specimens spiked at different LoDs (n=58 with at least 10 replicates for 1-2×LoD, 3-5×LoD, and pooled healthy donor saliva conditions). FIG. 2E shows schematic representation of the high-throughput, automatic assay realized by the BARA, demonstrating the use of 4 coverslips in parallel on a plate holder and an automated pipetting machine for simultaneous testing of 256 samples. FIG. 2F shows assessment of total fluorescence intensity of individual samples at various SARS-CoV-2 virion concentrations, ranging from 0 to 5×LoD. FIG. 2G shows interpretation of negative and positive samples after applying the cut-off value, resulting in a positive percentage agreement (PPA) of 100% and a negative percentage agreement (NPA) of 100%.

FIGS. 3A and 3B depict cross-reactivity, microbial interference, and endogenous/exogenous substances interference. FIG. 3A shows heatmap of antigen and nucleocapsid-encoding RNA signals during the evaluation of various respiratory pathogens, including 14 viruses, 15 bacteria, and 1 fungus at various concentrations (Table 2), in the presence of 0 and 3×LoD of SARS-CoV-2 virions with antigenic and nucleic acid detection for three replicates. FIG. 3B shows heatmap of spike glycoprotein and nucleocapsid-encoding RNA signals during the evaluation of various endogenous/exogenous substances, including 16 substances and 3 common antiviral and antibacterial medicines, spiked into healthy saliva samples at different concentrations (Table 3), in the presence of 0 and 3×LoD of SARS-CoV-2 virions with antigenic and nucleic acid detection for three replicates.

FIGS. 4A, 4B and 4C depict lineage and mutation monitoring with the BARA. FIG. 4A shows representative images of various SARS-CoV-2 variants, including the original Washington strain (USA-WA1/2020), alpha (B.1.1.7), beta (B.1.351), gamma (P.1), delta (B.1.617.2), omicron (BA.1) variants. Inset histograms illustrate the expression distributions of single virions as distributions of fluorescence intensity. FIG. 4B shows representative image of colocalized signals for spike glycoprotein, S2-encoding RNA, and the L452R mutation in a single virion of the delta variant. Additionally, colocalized signals for the spike glycoprotein, S2-encoding RNA, and the ΔH69 single point mutation in a virion of the omicron variant. FIG. 4C shows variant monitoring by detecting various Pango lineages of the omicron variant using the BARA for spike glycoprotein detection, including BA.5.1, BF.7, BQ.1, XBB.1.5, BA.2.12.1, and BA.2.3. Inset histograms illustrate the expression distributions of single virions as distributions of fluorescence intensity.

FIGS. 5A, 5B, 5C, 5D, 5E and 5F depict clinical validation of the BARA using saliva and NS specimens from COVID-19-positive patients. FIG. 5A shows antigenic and nucleic acid signals from COVID-19-positive patients are significantly enhanced in comparison to healthy donor (Mann-Whitney U test, ****p<0.0001, ***p<0.001) in both saliva (n=33 patients, n=30 healthy donors) and NS specimens (n=40 patients, n=19 healthy donors). FIG. 5B shows clinical validation of asymptomatic COVID-19-positive patient saliva (n=20 for asymptomatic, n=6 pooled healthy donor saliva). FIG. 5C shows correlation between antigenic and nucleic acid expression of saliva specimens, with minimal expression observed in the saliva of healthy donors (Pearson's correlation coefficient, p<0.0001 for r=0.76). FIG. 5D shows receiver operating characteristic (ROC) curves for combined detection of antigens and nucleic acids in salivary single SARS-CoV-2 virions with an enhanced area under the curve (AUC) value of 1.00 for dual detection. FIG. 5E shows correlation analysis of antigenic and nucleic acid expression of nasopharyngeal single SARS-CoV-2 virions, revealing a subpopulation of healthy donors with minimal expression of both biomarkers. FIG. 5F shows ROC curve analysis of antigenic, nucleic acid, and the dual biomarker expression in NS specimens, indicating an enhanced AUC value of 0.97.

FIGS. 6A, 6B, 6C, 6D and 6E depict virion-RNA detection in IHD-EVs of PASC patients. FIG. 6A shows comparison of virion-RNA content and the CD63 expression in single IHD-EVs compared to healthy donor plasma. FIG. 6B shows dual detection of CD63 and nucleocapsid-encoding RNA in single EVs of healthy donors and PASC patients P02 and P07. FIG. 6C shows colocalization analysis of CD63 and nucleocapsid-encoding RNA signals in single IHD-EVs. Inset demonstrates CD63+ IHD-EVs containing nucleocapsid-encoding RNA as highlighted by the white arrows. FIG. 6D shows detection of nucleocapsid-encoding RNA in thrombin-treated plasma of seven PASC patients (P01 to P07) at 3 serial timelines (T1, T2, and T3). A comparison with thrombin-treated plasma from donors who fully recovered from COVID-19 (HD13 and HD13*, indicating 1-month duration, HD44) and thrombin-treated plasma from a healthy donor (HD36, collected in 2019) prior to the COVID-19 outbreak. FIG. 6E shows virion-RNA loading percentage in CD63+IHD-EVs at different stages of infection.

FIGS. 7A and 7B depict Characterization and optimization of the BARA for detecting single SARS-CoV-2 virions. FIG. 7A shows single SARS-CoV-2 virions captured using different capture antibodies, including mouse-derived antibodies targeting the S1/S2 subunits of the spike glycoprotein, rabbit-derived antibodies targeting the S1/S2 subunits, antibodies targeting the SARS-CoV-2 membrane protein antibody, and recombinant ACE2 from R&D Systems and Sigma-Aldrich and detected for the spike glycoprotein (Table 1; n=2, error bars indicate the standard deviation). FIG. 7B shows scanning electron microscopy (SEM) images displaying single virions captured on the BARA, obtained through secondary electron detection. White arrows indicate the boundary between the bare gold surface and the antibody-coated inside the multiwell chamber area, where the antibody-coated area appears as a darker shade compared to the highly conductive gold surface. Black arrows indicate single SARS-CoV-2 virions captured on the antibody-coated surface.

FIGS. 8A, 8B and 8C depict cross-validation of spike glycoprotein and nucleocapsid-encoding RNA detection in SARS-CoV-2 virions using high-resolution flow cytometry. FIG. 8A shows scatter plots depicting spike glycoprotein detection on virions via high-resolution flow cytometry. The R1 region highlights the subpopulation of SARS-CoV-2 virions with a high fluorescence signal for the spike glycoprotein. FIG. 8B shows scatter plots showing the detection of SARS-CoV-2 nucleocapsid-encoding RNA using molecular beacons. The R2 region highlights the specific detection of the nucleocapsid-encoding RNA in SARS-CoV-2 virions compared to the murine leukemia virus (MLV) and negative control (phosphate-buffered saline; PBS). FIG. 8C shows comparison of particle counts in the R1 and R2 regions for the respective samples. All samples were measured at E7 particles/ml.

FIGS. 9A and 9B depict spatial characterization and dilution of a single SARS-CoV-2 virions with the BARA. FIG. 9A shows spatial expression of the spike glycoprotein and nucleocapsid-encoding RNA on a single SARS-CoV-2 virion, along with a cross-section of fluorescence intensity as a function of the x-y plane. FIG. 9B shows representative total internal reflection fluorescence microscopy (TIRFM) images showing different concentrations of virion particles captured with the BARA.

FIGS. 10A, 10B and 10C depict accelerated stability test for the BARA. FIG. 10A shows TIRFM images acquired over multiple days at 10° C. The white arrows indicate the individual SARS-CoV-2 virions detected. FIG. 10B shows temperature-induced degradation of the biochip and its corresponding reagents measured over 47 days at four elevated temperatures (10, 20, 25, and 30° C.), with results obtained at ten different time points (N=3, n=5, error bars indicate the standard deviation) FIG. 10C shows determination of the degradation rate constant of the biochip and its corresponding reagents at various temperatures using the Arrhenius equation and the methodology outlined in the Clinical and Laboratory Standards Institute (CLSI) EP25-A document. The calculated stability time (tstab) was 94.26 days at 4° C. for a 10% degradation.

FIGS. 11A and 11B depict genetic mutations on single SARS-CoV-2 virions with the BARA. FIG. 11A shows total fluorescence intensities of spike glycoprotein detection across variants and expression of ΔF157 and L452R mutations specific to the delta variant (n=2, error bars indicate the standard deviation). FIG. 11B shows total fluorescence intensities of six Pango lineages of the omicron variant for the spike glycoprotein and molecular beacons targeting the ΔH69 mutation specific to the omicron variant (n=2, error bars indicate the standard deviation).

FIGS. 12A and 12B depict sensitivity and specificity for COVID-19 diagnoses in saliva and nasopharyngeal swab (NS) specimens. FIG. 12A shows sensitivity and specificity for spike glycoprotein, nucleocapsid-encoding RNA, and dual detection using saliva specimens. FIG. 12B shows sensitivity and specificity for spike glycoprotein, nucleocapsid-encoding RNA, and dual detection using NS specimens.

FIGS. 13A and 13B depict Size distributions of SARS-CoV-2 virions and extracellular vesicles (EVs). FIG. 13A shows histogram representing the size distribution of extracellular vesicles (EVs) in healthy donor saliva specimens and purified by size-exclusion chromatography (SEC) measured using tunable resistive pulse sensing (TRPS). FIG. 13B shows histogram displaying the size distribution of SARS-CoV-2 virions measured by TRPS.

FIG. 14 depicts CoV-2 virion and EVs sorting with the BARA. TIRFM images illustrate specific capture and detection of subpopulations for SARS-CoV-2 virions and EVs from patient saliva samples. Antibodies targeting CD63/CD9 specifically captured CD63-expressing single EVs, whereas antibodies targeting the S1/S2 subunits of the spike glycoprotein enriched single SARS-CoV-2 virions expressing the spike glycoprotein.

FIGS. 15A, 15B and 15C depict EVs isolation from plasma. FIG. 15A shows representative TIRFM images showing EVs isolated from plasma using different isolation methods, including the total exosome isolation kit (TEI), thrombin, and SEC. FIG. 15B shows evaluation of CD63+ and CD81+ EVs isolated from plasma of a healthy donor using the various isolation methods (n=2, error bars indicate the standard deviation). FIG. 15C shows comparison of TRPS analysis between plasma samples treated with thrombin and SEC purification.

FIG. 16 describes assay workflow. A protein or RNA signal is considered positive if it is above the baseline (included in the Matlab algorithm). A sample is considered positive for COVID-19 if either protein or RNA signal is positive. A sample is considered negative for COVID-19 if both protein and RNA signals are negative.

FIG. 17 shows the assay controls-blank control: PBS, positive control: inactivated SARS-CoV-2 and internal control for each clinical sample: CD63 protein.

FIG. 18 describes data processing and analyzing workflow.

### DETAILED DESCRIPTION

Disclosed herein are methods for multiparametric detection of virus particles (for example, including extracellular vesicles) for the characterization of proteins and RNAs at the single nanoparticle level. The technology offers 100-fold more sensitivity than a traditional PCR or antibody assay.

Those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient.

The term “patient” refers to a subject under the treatment of a clinician, e.g., physician. The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “polymerase chain reaction” (“PCR”) refers to a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multiplex-PCR, reverse transcription PCR, or any of the other PCR variants known to those skilled in the art.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir.

The term “parenteral” includes subcutaneous, intravenous, intramuscular, intra-articular, intra-synovial, intrasternal, intrathecal, intrahepatic, intralesional, and intracranial injections or infusion techniques.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of disease), during early onset (e.g., upon initial signs and symptoms of disease), or after an established development of the disease. Prophylactic administration can occur for several days to years prior to the manifestation of symptoms of an infection.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule. As used herein, a “trinucleotide repeat” refers to a repetitive sequence of three base pair motifs in a DNA sequence. For example, the DNA sequence “GAAGAAGAAGAAGAA(n)” contains a repetitive sequence of GAA nucleotides, wherein n=any number. The trinucleotide repeat can be located in a coding or non-coding region of a genome.

A “nucleic acid” is a chemical compound that serves as the primary information-carrying molecules in cells and makes up the cellular genetic material. Nucleic acids are nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to nucleotide sequences, refer to the percentage of residue matches between at least two nucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known nucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined nucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

Spike protein” or “S” protein as used interchangeably herein refers to one of four main structural proteins of a coronavirus. The spike protein is heavily N-linked glycosylated and utilizes an N-terminal signal sequence to gain access to the endoplasmic reticulum (ER). Homotrimers of the virus-encoding S protein make up the distinctive spike structure on the surface of the virus. In many coronaviruses, the S protein is cleaved by a host cell furin-like protease into two separate polypeptides noted S1 and S2. S1 makes up the large receptor-binding domain (RBD) of the S protein while S2 forms the stalk of the spike molecule.

In some embodiments, high-throughput nano-biochip for high-efficiency, targeted EV capture and total internal reflective fluorescence microscopy (TIRFM) for rapid and high-resolution detection. A deep learning algorithm was developed to automate the analysis to acquire semiquantitative to quantitative information on the distribution of mRNA/miRNA and membrane proteins, as well as the colocation of multiple proteins and their ratios. The technology allows for rapid single-EV analysis and requires a very small sample quantity. This is the first technology that enables simultaneous detection and analysis of multiple types of biomolecules (e.g., nuclei acids and proteins) from both the surface and lumen of the EVs as biomarkers.

In one embodiment, “Total Internal Reflection Fluorescence Microscopy (TIRFM)” is used as a visualization technology. It is a specialized microscopy technique used to study fluorescence at the interface of two media, such as a glass coverslip and a liquid sample. Here, coating the glass with gold via a titanium intermediate layer aims to create a plasmonic surface to enhance fluorescence signals during TIRFM.

In some embodiments and claims, “plasmonic surface” refers to a surface that exhibits surface plasmon resonance (SPR). Surface plasmon resonance is a phenomenon that occurs when light interacts with free electrons at the interface between a dielectric material (such as glass or air) and a thin metal film (such as gold or silver). This interaction can enhance the electromagnetic field near the surface, leading to various optical and electronic effects.

In some embodiments and claims, “multiplex assay” refers to a laboratory technique that allows the simultaneous detection and measurement of multiple analytes (such as proteins, nucleic acids, or other molecules) in a single experimental run. This simultaneous analysis of multiple targets provides several advantages over traditional singleplex assays, including increased efficiency, reduced sample consumption, and the ability to gain comprehensive information from a single sample.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Methods

In some aspects, disclosed herein a method of detecting a viral antigen and a viral nucleic acid in a subject, comprising:
obtaining a biological sample from the subject;
capturing a virion from the biological sample; or
capturing a membranous particle from the biological sample; and
measuring the viral antigen and the viral nucleic acid level in the biological sample;
wherein the virion or membranous particle is immobilized on a biochip.

In some embodiments, the biochip comprises of a plasmonic surface.

In some embodiments, the viral nucleic acid comprises RNA. In some embodiments, the viral nucleic acid comprises DNA.

In some embodiments, the viral antigen comprises a viral protein.

In some embodiments, the viral antigen or viral nucleic acid is detected when the viral antigen and the viral nucleic acid levels are increased in comparison to a control sample.

In some embodiments, the virion comprises a coronavirus virion, an influenza virus virion, or a respiratory syncytial virus (RSV) virion.

In some embodiments, the membranous particle comprises a virion-infected host-derived extracellular vesicle.

In some aspects, disclosed herein a method of treating a viral infection in a subject, comprising:
obtaining a biological sample from the subject;
capturing a virion from the biological sample; or
capturing a membranous particle from the biological sample;
measuring a viral antigen or a viral nucleic acid level in the biological sample;
wherein the virion or membranous particle is immobilized on a biochip; and
administering an antiviral agent if a viral infection is detected.

In some embodiments, the biochip comprises a plasmonic surface.

In some embodiments, the viral infection is detected when the viral antigen or nucleic acid level is increased in comparison to a control sample.

In some aspects, disclosed herein a method for high throughput multiplexing for simultaneous detection of a viral antigen and a viral nucleic acid, the method comprising the steps of:
immobilizing a virion and/or a membranous particle on a plasmonic surface;
detecting the viral antigen and the viral nucleic acid simultaneously derived from the virion and/or membranous particle;
wherein the detection method comprises fluorescence, in situ hybridization, enzyme linked immunosorbent assay, flow cytometry, or microscopy.

In some embodiments, the viral antigen and the viral nucleic acid are detected with a sensitivity of at least 80%.

In some embodiments, the viral antigen and the viral nucleic acid are detected with a specificity of at least 95%.

In some aspects, disclosed herein is a method of detecting a coronavirus, comprising: obtaining a biological sample;
capturing a plurality of membranous particles from the biological sample;
measuring a spike(S) protein level in the membranous particles from the biological sample; and
measuring an amount of a coronavirus RNA;
wherein a coronavirus is detected when the spike protein level or the amount of coronavirus RNA is increased in comparison to a control sample.

In some embodiments, a coronavirus is detected when the spike protein level and the amount of coronavirus RNA is increased in comparison to a control sample.

In some embodiments, the coronavirus is SARS-CoV-2.

In some embodiments, the biological sample is saliva. In some embodiments, the biological sample is a nasopharyngeal swab. In some embodiments, the biological sample is a plasma sample.

In some embodiments, the RNA is measured using one or more probes complementary to a target nucleic acid sequence. In some embodiments, the RNA is measured using one or more probes complementary to a target RNA sequence.

In some embodiments, the membranous particles at coronavirus particles. In some embodiments, the membranous particles are extracellular vesicles.

In some embodiments, the method further comprises administering an antiviral agent if a coronavirus is detected when the spike protein level or the amount of SARS-CoV-2 RNA is increased in comparison to a control sample.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Integrated Antigenic and Nucleic Acid Detection in Single Virions and Virion-Infected Host-Derived Extracellular Vesicles

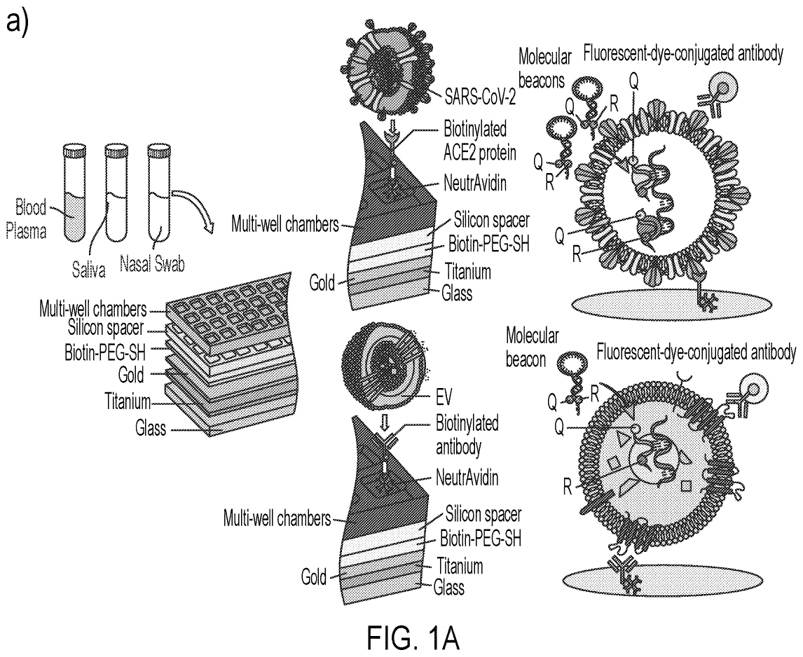

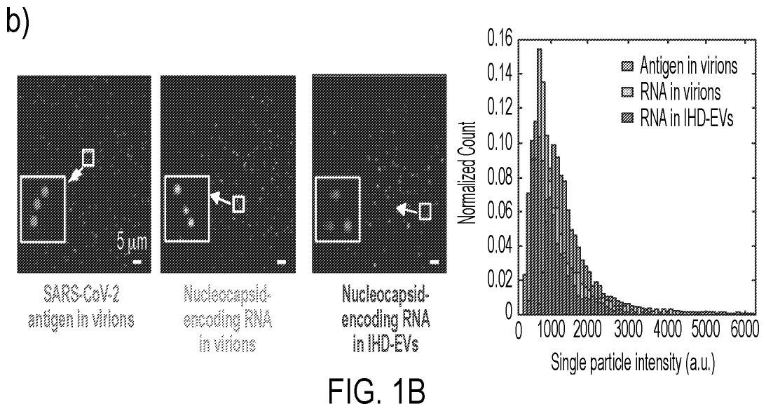

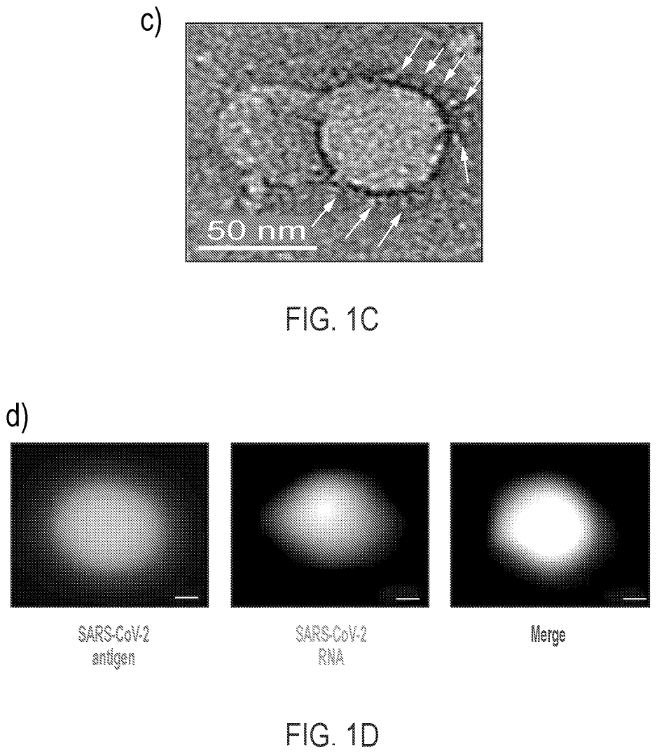

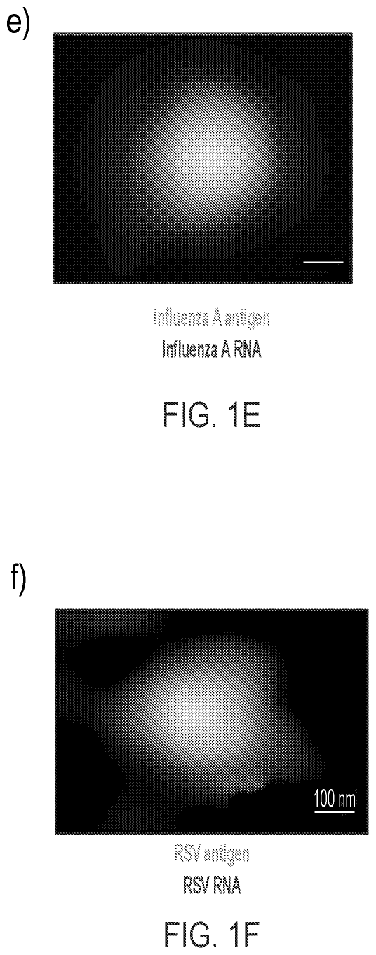

Simultaneous detection of biomolecules on intact single virions and EVs. The BARA is a high-throughput assay that multiplexes signals from antigens and nucleic acids utilizing IF and FISH on single virions and IHD-EVs derived from complex biofluids, such as blood plasma, saliva, and NS. Briefly, glass is coated with gold via a titanium intermediate, providing a plasmonic surface to enhance fluorescence signals emitted by TIRFM. The gold surface is reacted with thiol-poly(ethylene glycol)-biotin enabling the subsequent functionalization of NeutrAvidin (NA) and biotinylated antibodies and proteins targeting external epitopes of the single particles. Lastly, fluorescent-dye-conjugated antibodies were utilized to perform IF, and molecular beacons were utilized to perform FISH (FIG. 1A). TIRFM provides an evanescent wavefront that exponentially decreases from the coverslip surface, affording the visualization of biomolecules in single virions and IHD-EVs as localized fluorescent signals that can be quantified as functions of fluorescent emissions (FIG. 1B). As a model system, SARS-CoV-2 was chosen for the validation of the BARA. Transmission electron microscopy (TEM) revealed the presence of spike glycoproteins forming the corona that is a hallmark of coronaviruses (FIG. 1C). Therefore, the spike glycoprotein along with targeting multiple regions of the nucleocapsid-encoding RNA via combining IF and FISH provided the colocalization of fluorescent signals on a single-localized domain, providing evidence for the co-expression of biomolecules on a single virion (FIG. 1D). While SARS-CoV-2 is the model system for validation, the tunable nature of the BARA is illustrated by simultaneous targeting antigens and nucleic acids for Influenza A (FIG. 1E) and the respiratory syncytial virus (RSV; FIG. 1F). The BARA provides a tunable platform to multiplex antigenic and nucleic acid signals in single virions and IHD-EVs.

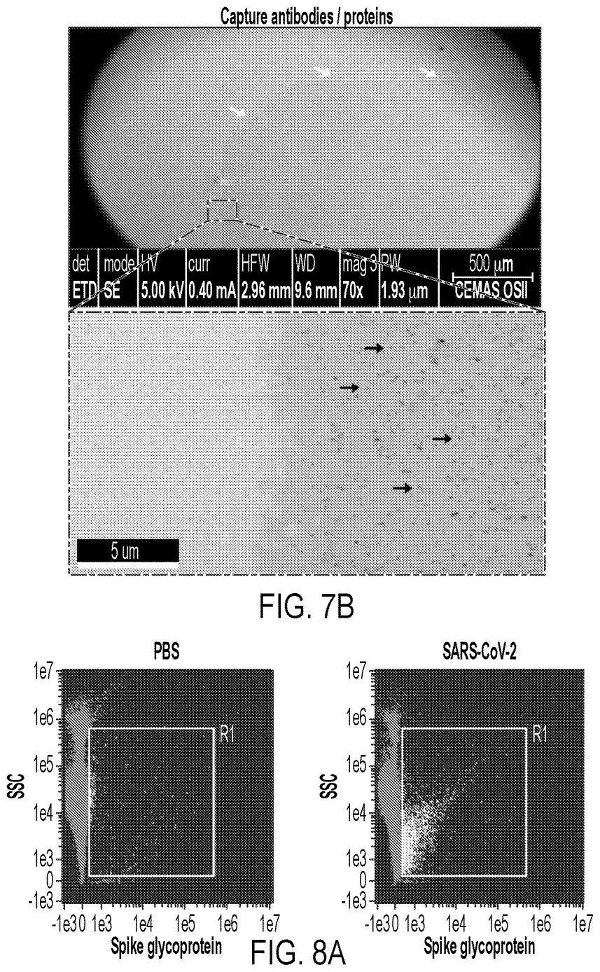

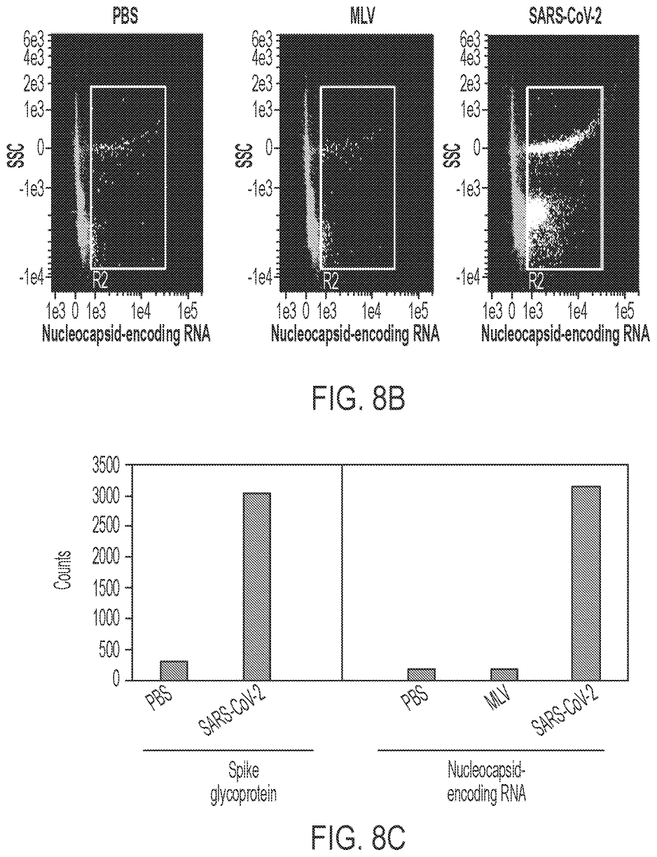

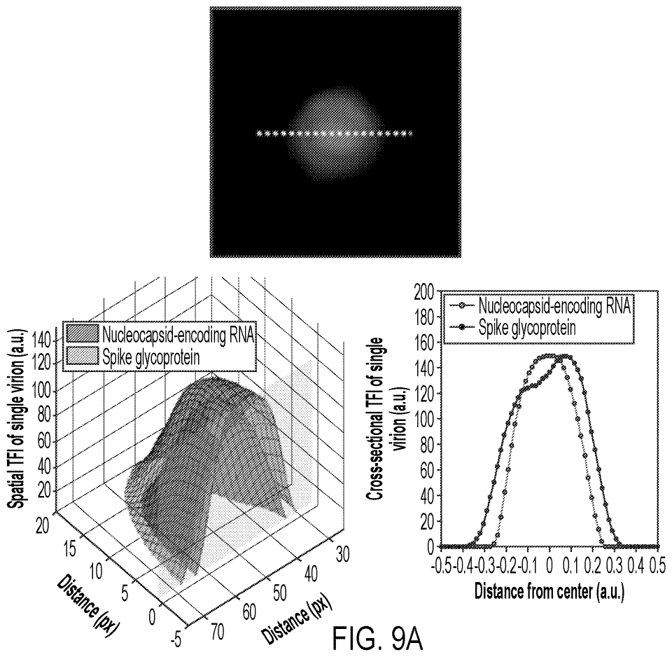

Specific and sensitive detection of single SARS-CoV-2 virions. Single SARS-CoV-2 virions were isolated onto the plasmonic surface of the BARA, then were tested using different methods for immunopositive selection, including antibodies targeting the S1 and S2 subunits of the spike glycoprotein and the coronavirus membrane protein. On the other hand, immunopositive selection was also performed by simulating the port of entry for cellular infection with recombinant angiotensin-converting enzyme 2 (ACE2). Optimizing the surface for the capture of single virions, demonstrated that targeting the spike glycoprotein via antibodies or recombinant ACE2 provided the highest relative fluorescence intensity for IF of the spike glycoprotein (FIG. 7A, Table 1). Relative fluorescence intensity was defined as the sum of fluorescence intensity signals of the sample normalized by that of the negative control (phosphate-buffered saline, PBS). Scanning electron microscopy demonstrated the capture of single SARS-CoV-2 virions within the ACE2-functionalized surface of the BARA (FIG. 7B). Flow cytometry was performed on spike glycoprotein detection for SARS-CoV-2 virions and PBS to cross-validate the IF of the spike glycoprotein with the BARA, revealing a fluorescent enrichment for the sample only (FIG. 8A). For nucleic acid detection, specificity was tested with flow cytometry by targeting three regions of the nucleocapsid-encoding RNA on SARS-CoV-2, murine leukemia virus (MLV), and PBS. The molecular beacons hybridized at higher rates for the SARS-CoV-2 samples, whereas MLV and PBS yielded similarly low levels of fluorescent signal (FIG. 8B). Having detection methods for antigens and nucleic acids in SARS-CoV-2 virions were tested to colocalize signals by combining detection methods in single virions. Therefore, the BARA while co-targeting the spike glycoprotein and nucleocapsid-encoding RNA via in situ TIRFM image acquisition was utilized. Colocalized signals were observed and investigated multi-dimensionally. Apart from measuring fluorescence intensity one-dimensionally as total or relative fluorescence intensities, herein demonstrated is the spatial expression of the spike glycoprotein and nucleocapsid-encoding RNA on a single SARS-CoV-2 virion by measuring fluorescence intensity as a function of the x-y plane (FIG. 9A). Furthermore, cross-sections of the three-dimensional expression provide a two-dimensional fluorescence intensity profile as a function of an axis, revealing more spatially variable and diffuse expression of the spike glycoprotein compared to the nucleocapsid-encoding RNA (FIG. 9A).

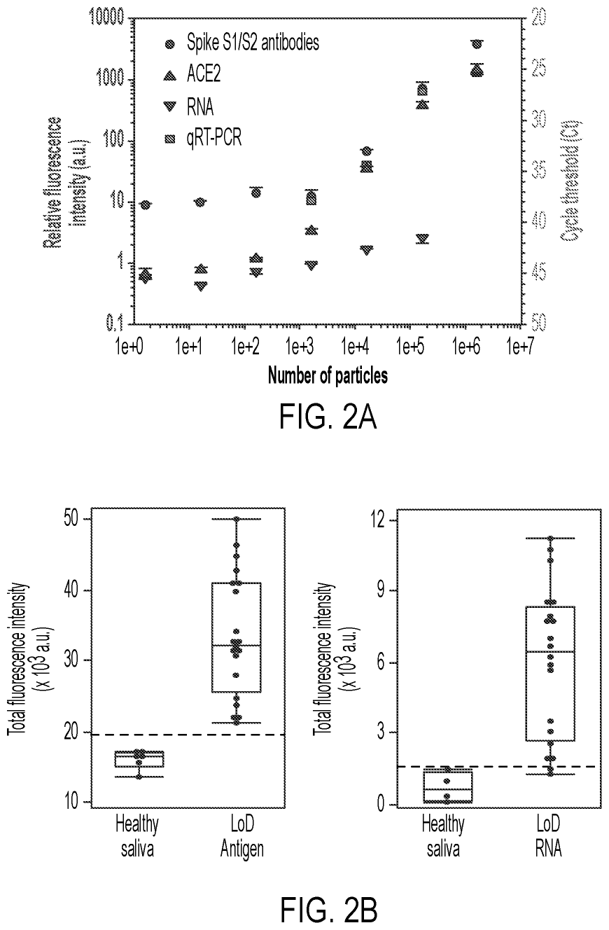

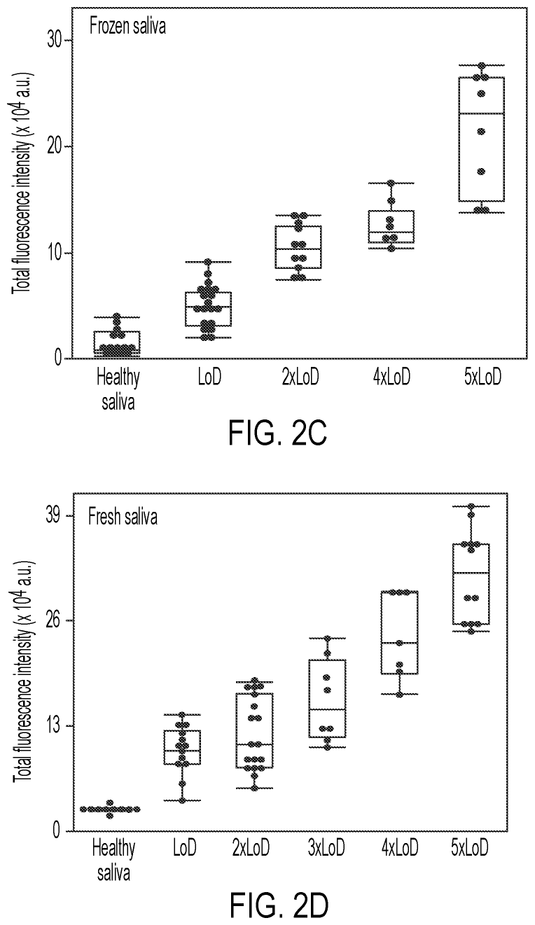

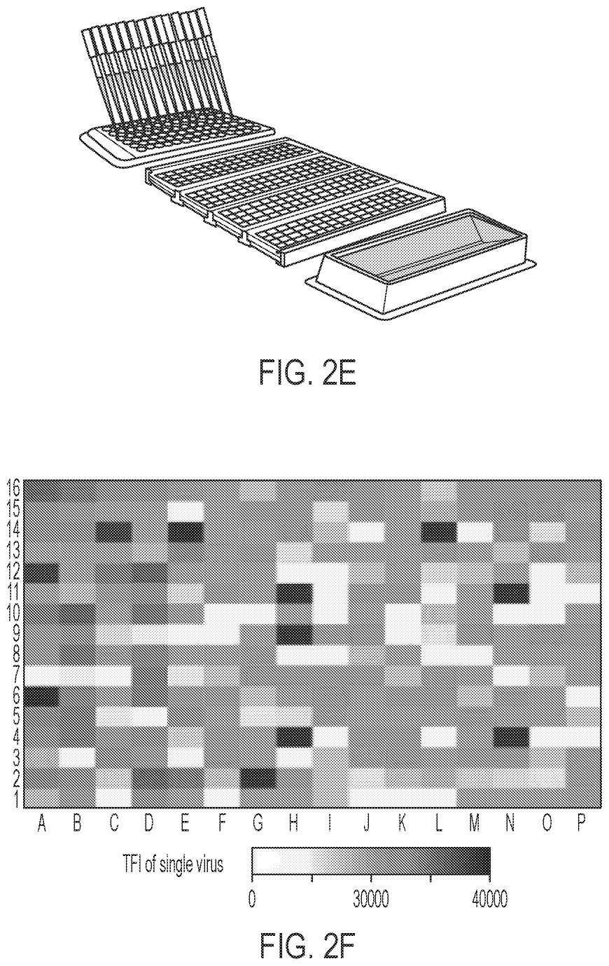

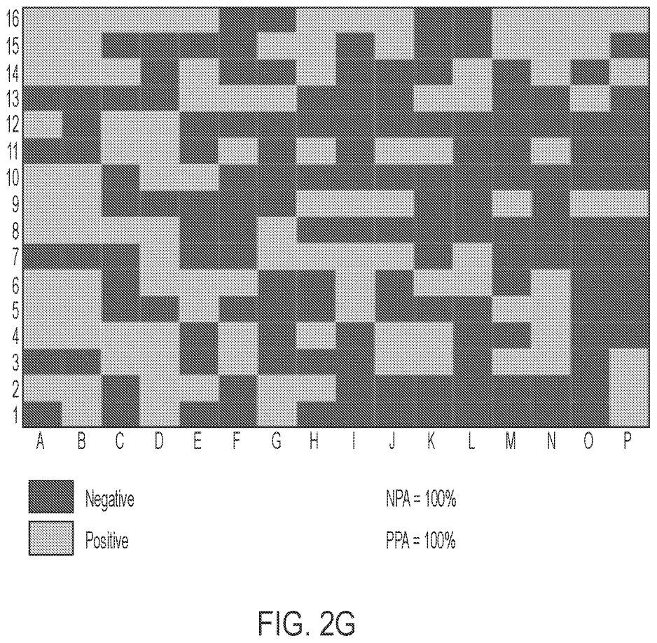

Demonstrating an ability to distinguish antigenic and nucleic acid signals in single SARS-CoV-2 virions from negative controls, the sensitivities of the BARA was characterized. Therefore, a comparison was done among the BARA utilizing the top candidates for virion capture to the most sensitive COVID-19 diagnostic assay, qRT-PCR. Targeting S1 and S2 subunits of the spike glycoprotein exhibited a linear range of 10\hat{ }3-10\hat{ }6 particles/well (R2=0.99; ANOVA, p=0.0023), whereas utilizing recombinant ACE2 to immobilize the single virions lent a linear range of 10\hat{ }2-10\hat{ }6 particles/well (R2=0.98; ANOVA, p=0.0008) when detecting the spike glycoprotein (FIG. 2A, FIG. 9B). Similarly, detecting nucleocapsid-encoding RNA when immobilizing with recombinant ACE2 demonstrated a linear range of 10\hat{ }2-10\hat{ }6 particles/well (R2=0.97; ANOVA, p=0.0152). Therefore, both SARS-CoV-2 antigenic and nucleic acid detection via ACE2-mediated immobilization outperformed qRT-PCR by an order of magnitude, which became undetectable at 10\hat{ }2 particles/well (FIG. 2A). To further demonstrate the ability of the BARA to detect at the limit of detection (LoD), ACE2-mediated immobilization was coupled with both spike glycoprotein and nucleocapsid-encoding RNA to detect SARS-CoV-2 virions spiked into the healthy donor saliva at 10\hat{ }2 particles/well. Compared to healthy donor saliva, the single SARS-CoV-2 virions at the LoD demonstrated a significantly higher signal for spike glycoprotein detection (FIG. 2B; Welch's two-tailed t-test, p<0.0001). Furthermore, detection of the nucleocapsid-encoding RNA at the LoD further demonstrated significantly higher total fluorescence intensities than healthy saliva (FIG. 2C; Welch's two-tailed t-test, p<0.0001). Next, to test whether freezing saliva affected the ability to detect the spike glycoprotein with the BARA. Therefore, SARS-CoV-2 virions were spiked at varying dilutions into saliva and tested immediately or frozen then rethawed. While there was an effect of freezing on the detection of various dilutions, such as a reduction in the slope (ANOVA, p<0.0001 for the interaction effect), the BARA could discern the dilutions linearly for both frozen and fresh saliva samples (FIG. 2C-D; ANOVA, p=0.0090 for frozen saliva and p=0.0003 for fresh saliva). To further show the utility of the BARA as an automated high-throughput diagnostic assay, four BARA assays are assembled in parallel alongside an automated pipetting machine for the facile testing of 256 samples. Various dilutions of SARS-CoV-2 virions were introduced, allowing for the testing of 256 samples at different LoD simultaneously (FIG. 2E). Utilizing the high-throughput technique, each sample corresponded with a total fluorescence intensity (FIG. 2F), which is translated to SARS-CoV-2 positivity via higher total fluorescence intensities than that of the LoD (FIG. 2G). With this cutoff, the BARA yielded a positive percentage agreement (PPA) of 100% and a negative percentage agreement (NPA) of 100% (FIG. 2G).

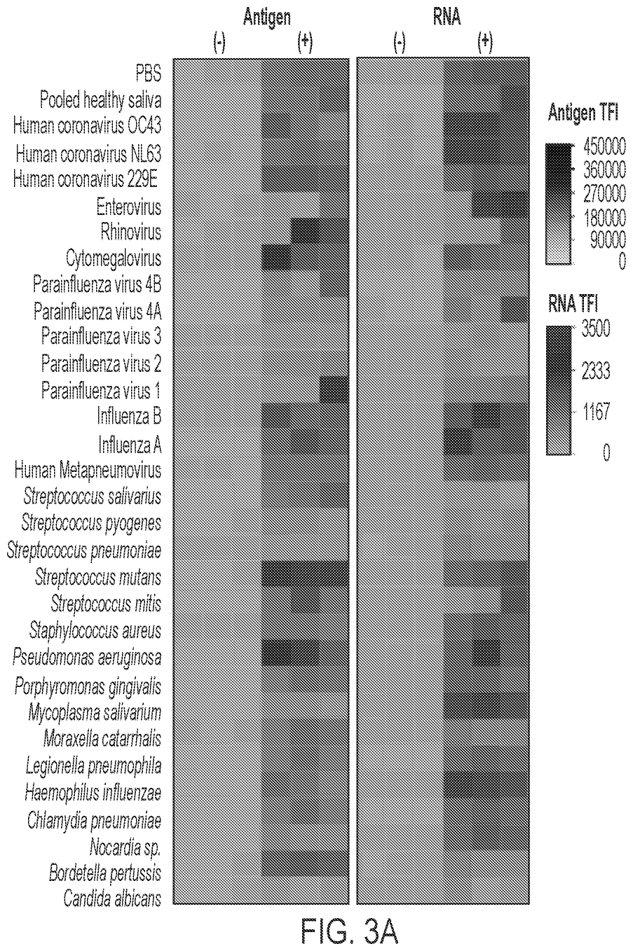

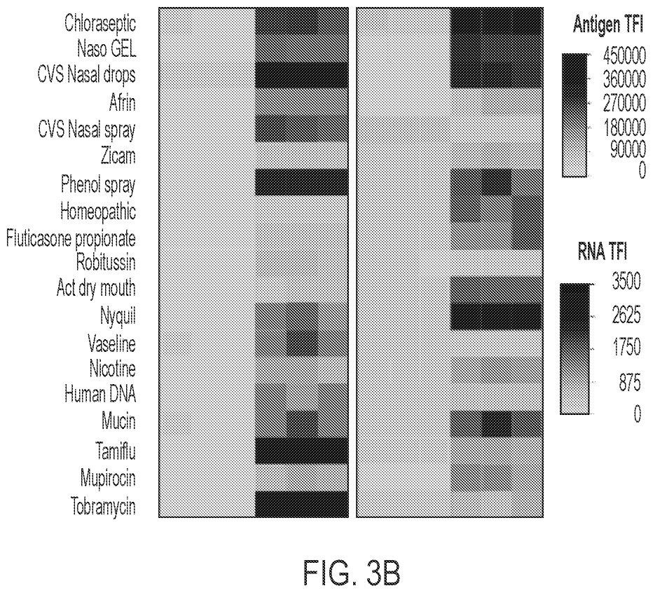

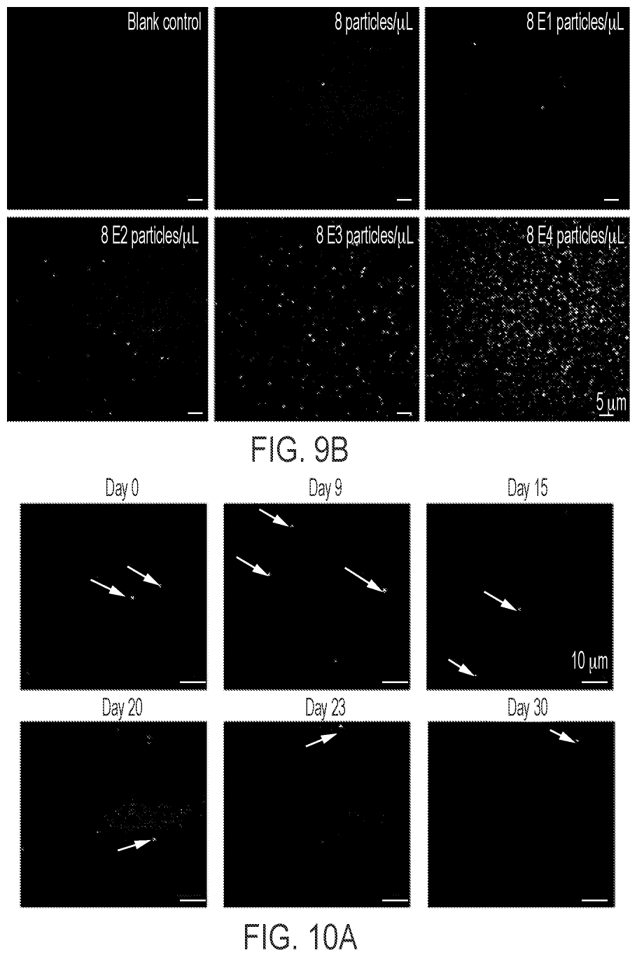

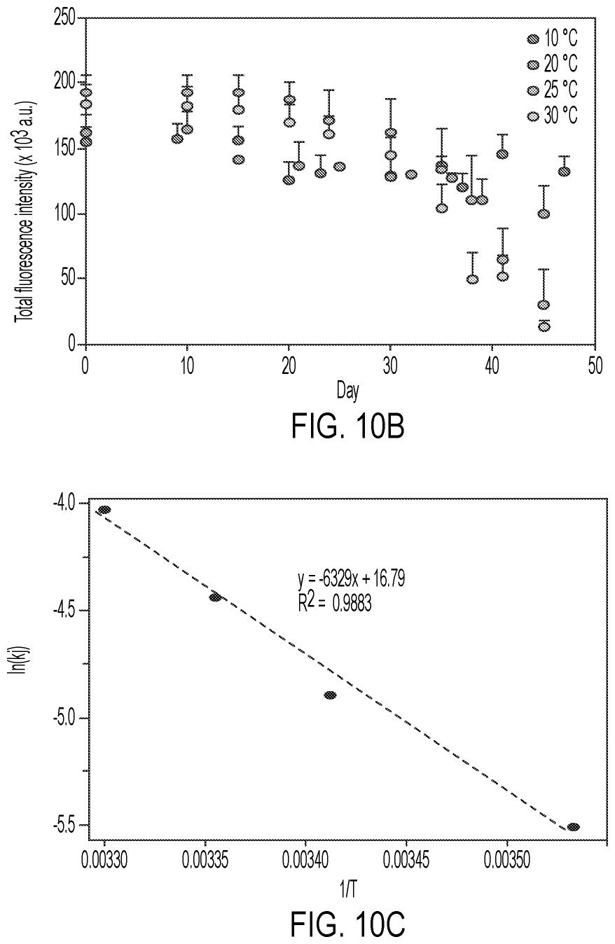

The LoD of SARS-CoV-2 virions was tested three times for antigenic and nucleic acid detection with the BARA in the presence of multiple respiratory pathogens (Table 2). While the respiratory pathogens affected the fluorescent signal (ANOVA, p<0.0001 for the interaction effect for both antigenic and nucleic acid detection), the BARA accurately discriminated between samples spiked with the SARS-CoV-2 virions and the corresponding control (FIG. 3A; ANOVA, p<0.0001 for the effect of spiking for both antigenic and nucleic acid detection). Furthermore, three times the LoD in the presence of various endogenous and exogenous substances used for curing or lessening symptoms associated with SARS-CoV-2 infections was tested with the BARA (Table 3). Despite the endogenous and exogenous substance utilized affecting the fluorescence intensity (ANOVA, p<0.0001 for the interaction effect for both antigenic and nucleic acid detection), the BARA distinguished the spiked samples from the control in the presence of endogenous and exogenous substances (FIG. 3B; ANOVA, p<0.0001 for the effect of spiking for both antigenic and nucleic acid detection). Lastly, to test the ability to disseminate the BARA, accelerated stability was performed according to the Clinical and Laboratory Standards Institute (CLSI) EP25-A to evaluate the stability of in vitro diagnostic reagents (Table 4-5). The entire assay, including the biochip and the reagents therein, were incubated and tested at various temperatures and time points to determine the efficiency of detecting the spike glycoprotein on single SARS-CoV-2 virions (FIG. 10A). Utilizing the Arrhenius equation, various rate constants for each temperature were extracted from which the rate of degradation at 4° C. was extrapolated (FIG. 10B-C), revealing a 10% degradation of the BARA at 94.26 days when stored at 4° C. Therefore, the BARA holds diagnostic promise as an automated high-throughput clinically relevant assay for simultaneously detecting antigens and nucleic acids in single SARS-CoV-2 virions with sensitivities higher than qRT-PCR.

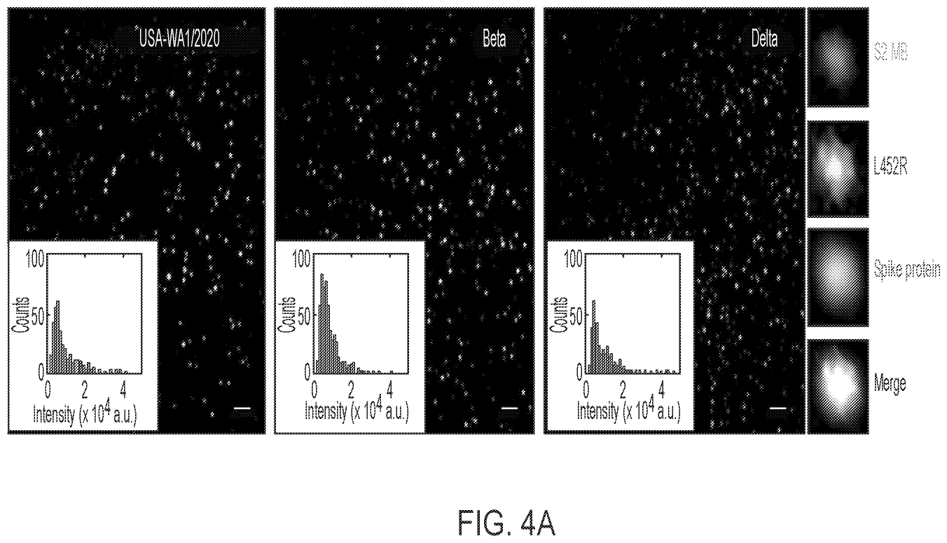

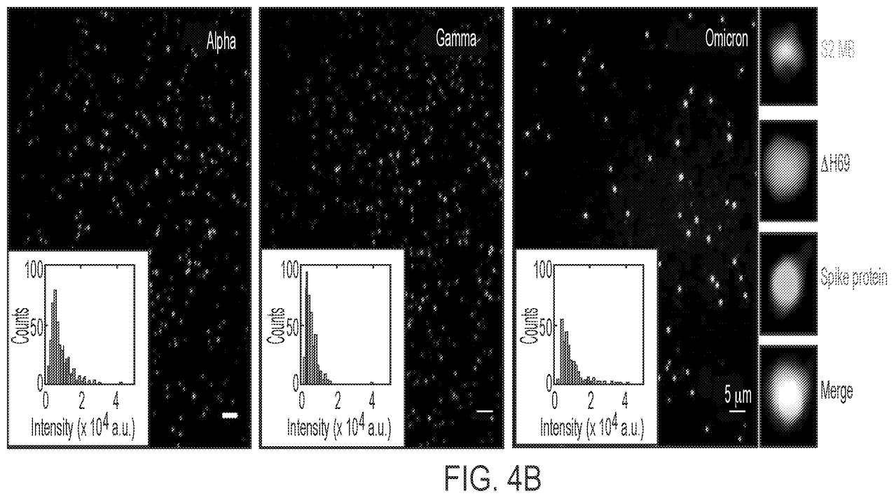

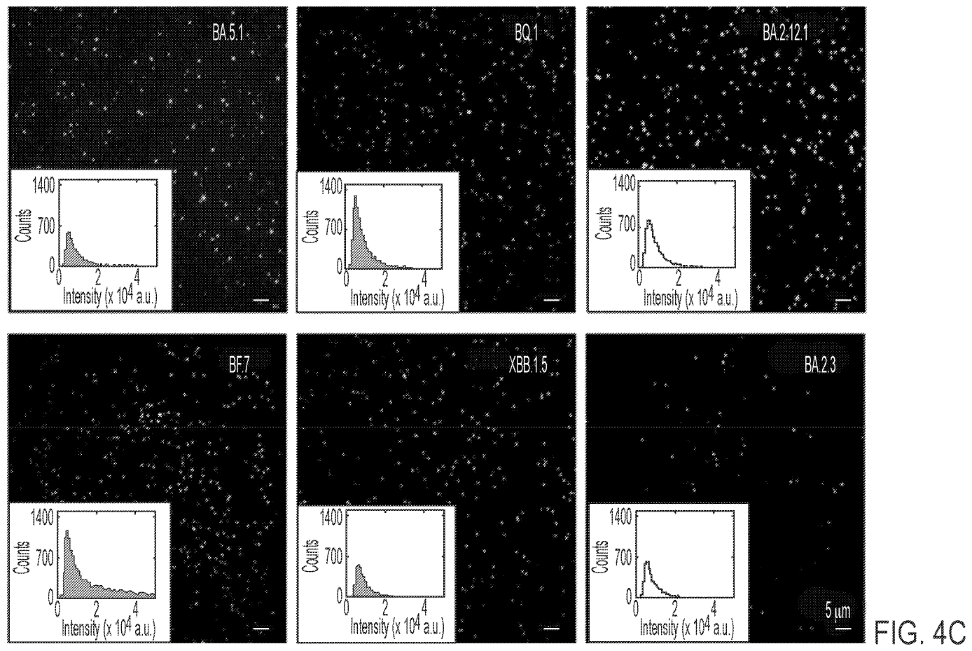

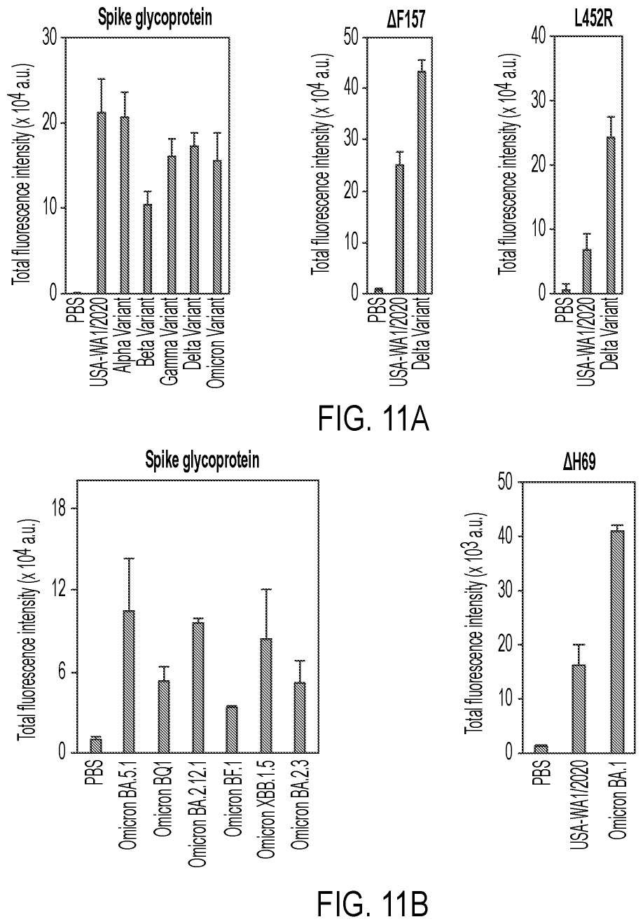

Monitoring genetic mutations on single virions. Given that SARS-CoV-2 variants alter the structure of the spike glycoprotein to evade immune responses, the ability of the BARA to detect the spike glycoprotein on the various variants experienced during the pandemic in the USA, including the original Washington strain (USA-WA1/2020), alpha, beta, gamma, delta, and omicron strains was tested. Despite the mutations, positive signals for the spike glycoprotein were obtained for all strains (FIG. 4A). Furthermore, the BARA distinguishes positive signals for all strains from the negative control of PBS (FIG. 11A; Dunnett's test, p≤0.0162). Since variants arise from mutations in the viral genome, to test whether the enhanced sensitivity of the BARA is sufficient to detect genetic mutations at a single-virion resolution. Therefore, molecular beacons were designed to co-target S2-encoding RNA and variant-specific mutations for delta and omicron variants emitting different wavelengths upon TIRFM excitation. To determine whether the BARA discerns the ΔF157 and L452R mutations present in the delta variant from the Washington strain. The ΔF157 mutations revealed signal enrichment for the delta variant as opposed to the original Washington strain (Tukey's HSD, p=0.0045). However, the Washington strain emitted a higher total fluorescence intensity than the control (FIG. 11A; Tukey's HSD, p=0.0018). On the other hand, the L452R mutation was overexpressed in the delta strain as opposed to the original Washington strain (Tukey's HSD, p=0.0106) and was absent for the Washington strain (FIG. 11A; Tukey's HSD, p=0.3309). Therefore, for the delta variant, the BARA colocalized signals for the spike glycoprotein, S2-encoding RNA, and the L452R mutation whereby the colocalization of fluorescent signals as the primary colors of light added to white light, demonstrating the co-expression of the biomolecules on a single virion (FIG. 4B). Similarly, the BARA colocalized signals for the spike glycoprotein, S2-encoding RNA, and the ΔH69 mutation on single virions (FIG. 4B), revealing an enrichment in the omicron variant when compared to the original Washington strain (FIG. 11B; Tukey's HSD, p=0.0018), albeit detectable in the original Washington strain (Tukey's HSD, p=0.0085). While the BARA distinguished positive signals for the different variants, to further determine the robustness of spike glycoprotein detection, multiple Pango lineages of the omicron variant were tested, including BA.5.1, BF.7, BQ.1, XBB.1.5, BA.2.12.1, and BA.2.3. Positive signals were observed for the Pango lineages (FIG. 4C) and were significantly higher than the negative control for BA.2.12.1, BA.5.1, and XBB.1.5 (FIG. 11B; Dunnett's test, p≤0.0487).

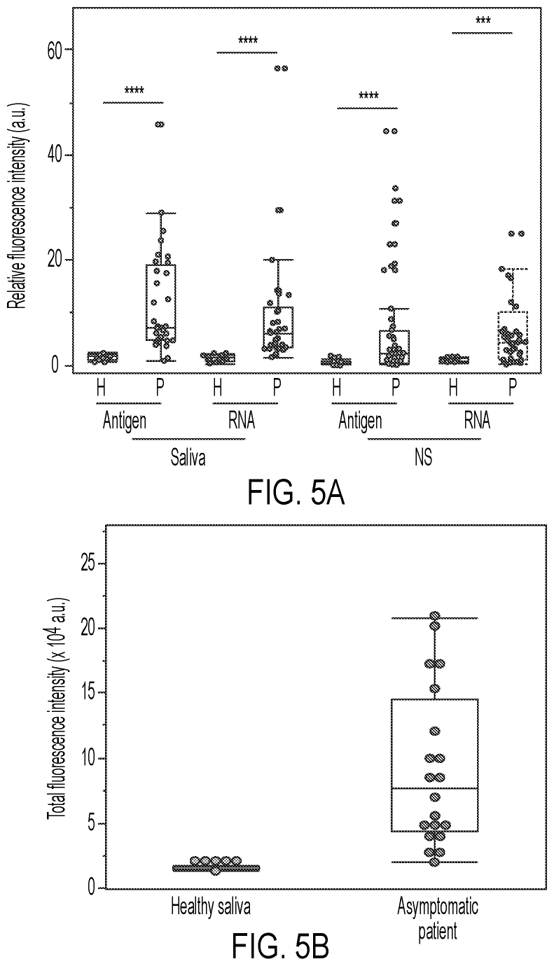

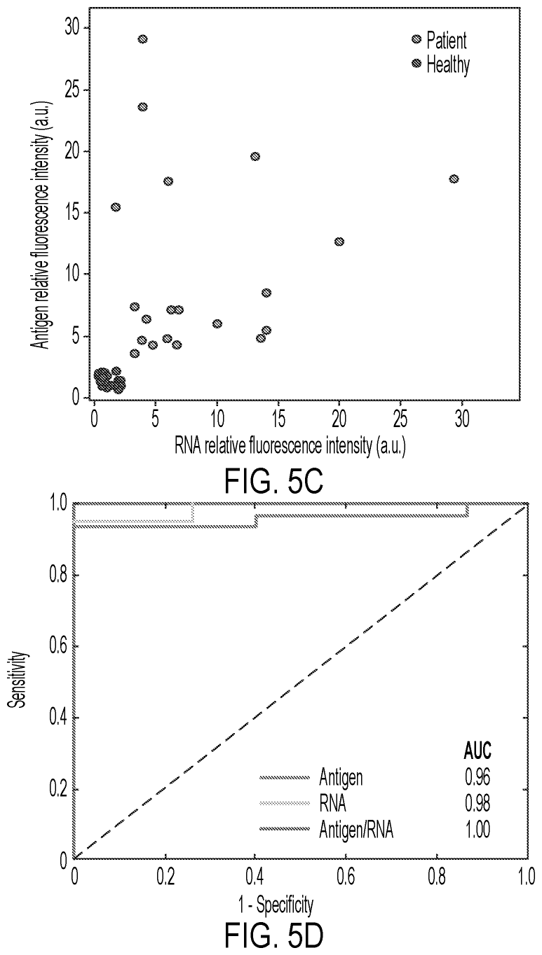

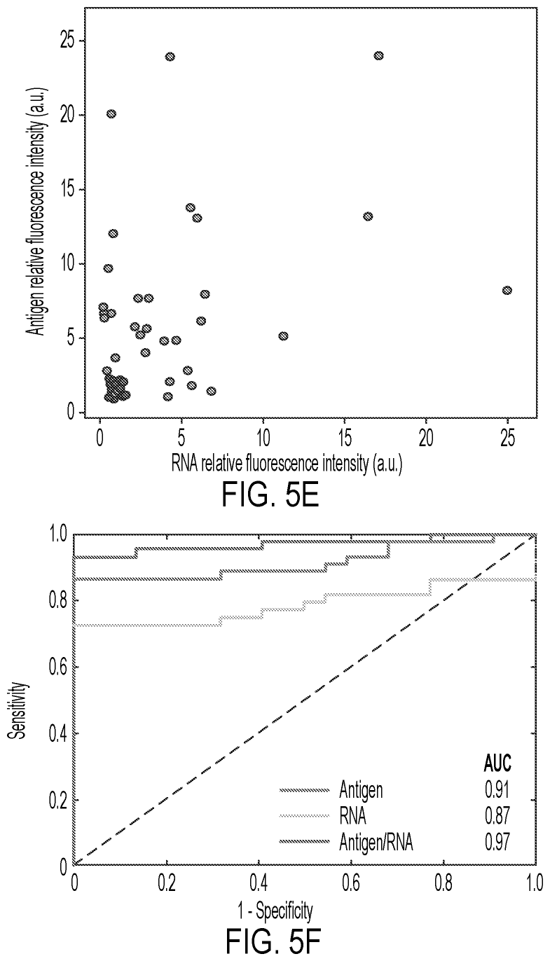

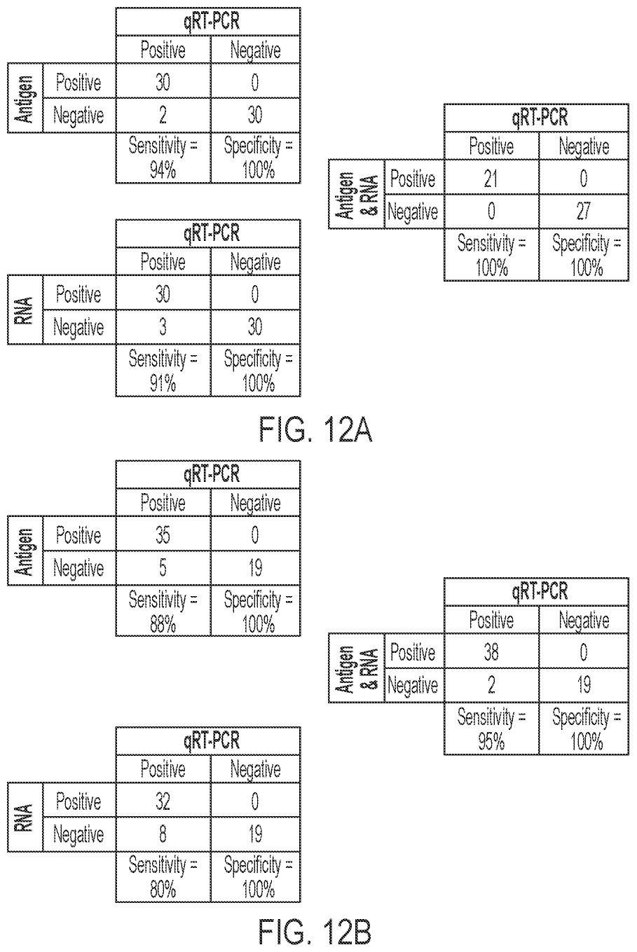

Dual antigenic and nucleic acid detection for COVID-19 diagnosis. To test and validate the clinical potential of the BARA for virion-mediated infections, its use for distinguishing COVID-19 patients utilizing complex biofluid samples, including saliva (n=33 patients, n=30 healthy donors) and NS (n=40 patients, n=19 healthy donors). Based on clinical testing, patient specimens were collected from PCR-confirmed COVID-19 cases (Table 6-7). Spike glycoprotein and nucleocapsid-encoding RNA were utilized to determine COVID-19-positive patients. Both antigenic and nucleic acid detection on single SARS-CoV-2 virions demonstrated an enhanced signal for COVID-19 patients for saliva (FIG. 5A; Mann-Whitney U test, p<0.0001 for antigenic and nucleic acid detection) and NS (FIG. 5A; Mann-Whitney U test, p<0.0001 for antigenic detection, p=0.0002 for nucleic acid detection). Specifically, spike glycoprotein detection in salivary single SARS-CoV-2 virions revealed a sensitivity of 94% and a specificity of 100%, whereas nucleocapsid-encoding RNA detection yielded a sensitivity of 91% and a specificity of 100% (FIG. 12A). On the other hand, detection of nasopharyngeal single SARS-CoV-2 virions provided sensitivities of 88% and 80% for spike glycoprotein and nucleocapsid-encoding RNA detection, respectively, and specificities of 100% for both detection methods (FIG. 12B). Given that the highest specificity was obtained for spike glycoprotein detection in salivary single SARS-CoV-2 virions, and it was analyzed whether the enhanced sensitivity of the single-virion method diagnose the COVID-19 asymptomatic patient subpopulation. Therefore, a cohort of 20 asymptomatic patients was tested with the BARA, revealing an enrichment of signals for the asymptomatic cohort compared to healthy donors (FIG. 5B; Mann-Whitney U test, p=0.0003).

To further improve the sensitivities of the BARA as NS demonstrated lower diagnostic performances, the BARA was utilized to dual detect antigenic and nucleic acid signals to provide a combinatorial approach to diagnose COVID-19 for salivary and nasopharyngeal samples. Antigenic and nucleic acid expression strongly coincided (Pearson's correlation coefficient, p<0.0001 for r=0.76) and had weak negative associations with qRT-PCR cycle-threshold values for COVID-19 patients (Pearson's correlation coefficient, r=−0.33 for antigenic detection, r=−0.14 for nucleic acid detection), revealing a distinct subpopulation for healthy donors with minimal expression of both biomarkers (FIG. 5C). Utilizing receiver operating characteristic (ROC) curves, the combined detection of antigens and nucleic acids in salivary single SARS-CoV-2 virions augmented the area under the curve (AUC) from 0.96 for antigenic detection and 0.98 for nucleic acid detection to 1.00 for dual detection (FIG. 5D). Thus, the sensitivity was enhanced to 100% (FIG. 12B). Despite the expression of nasopharyngeal single SARS-CoV-2 virions only weakly associating between antigenic and nucleic acid detection (Pearson's correlation coefficient, r=0.15) with weak negative correlations with qRT-PCR cycle-threshold values for COVID-19 patients (Pearson's correlation coefficient, r=−0.29 for antigenic detection, r=−0.44 for nucleic acid detection), a subpopulation of healthy donors with minimal expression of both biomarkers was present with slight intercalations of COVID-19 patients (FIG. 5E). ROC curves revealed the enhanced diagnostic capability of the BARA by utilizing the combinatorial method, increasing the AUC from 0.91 and 0.87 for single antigenic and nucleic acid detection, respectively, to 0.97 for dual detection (FIG. 5F), which increased the sensitivity to 95% (FIG. 12B). Therefore, dual antigenic and nucleic acid detection with the BARA enhances sensitivities for a reliable COVID-19 diagnostic assay.

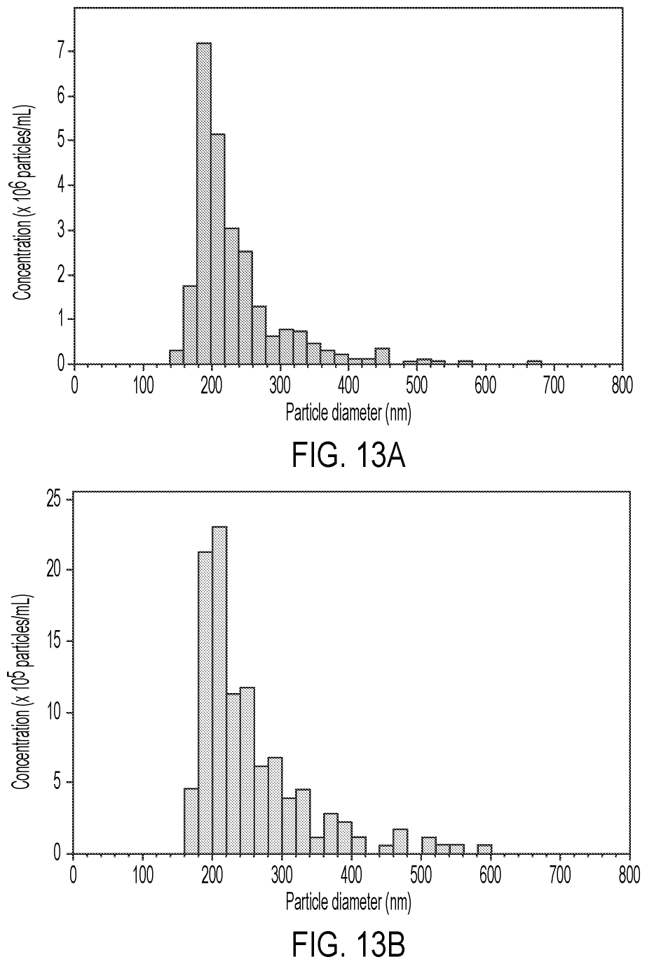

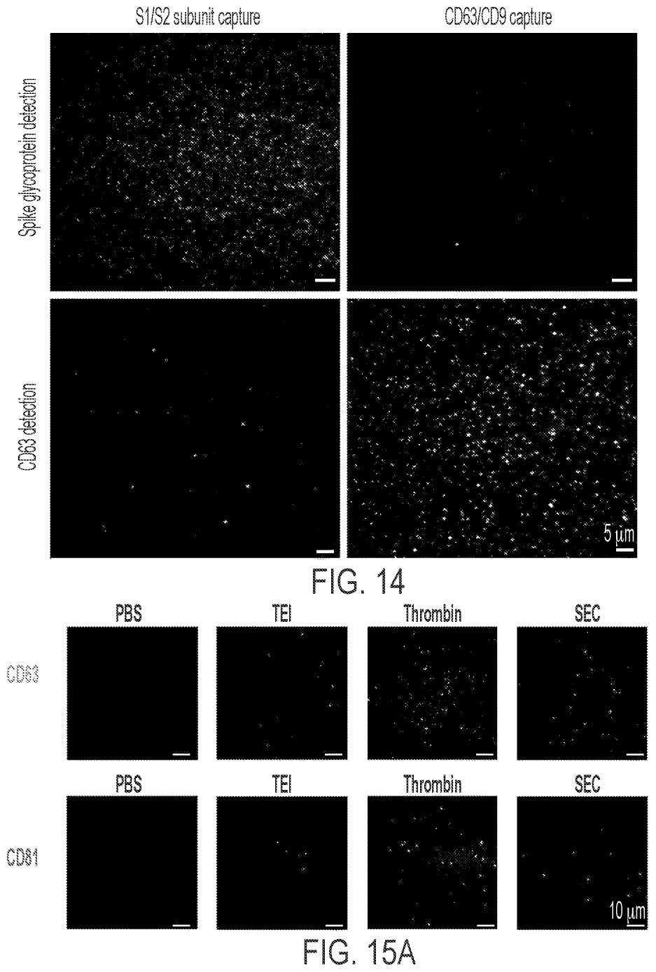

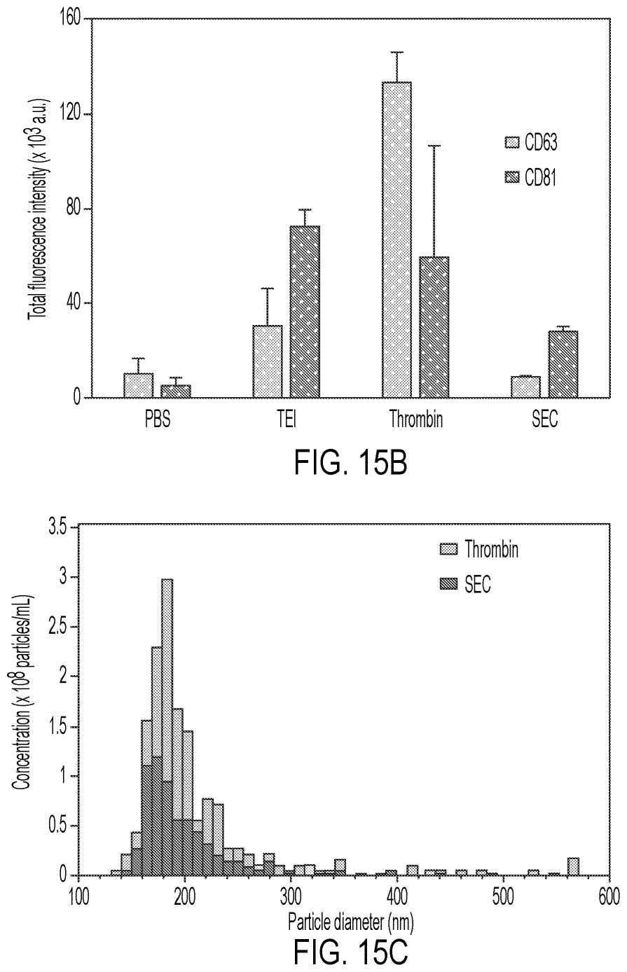

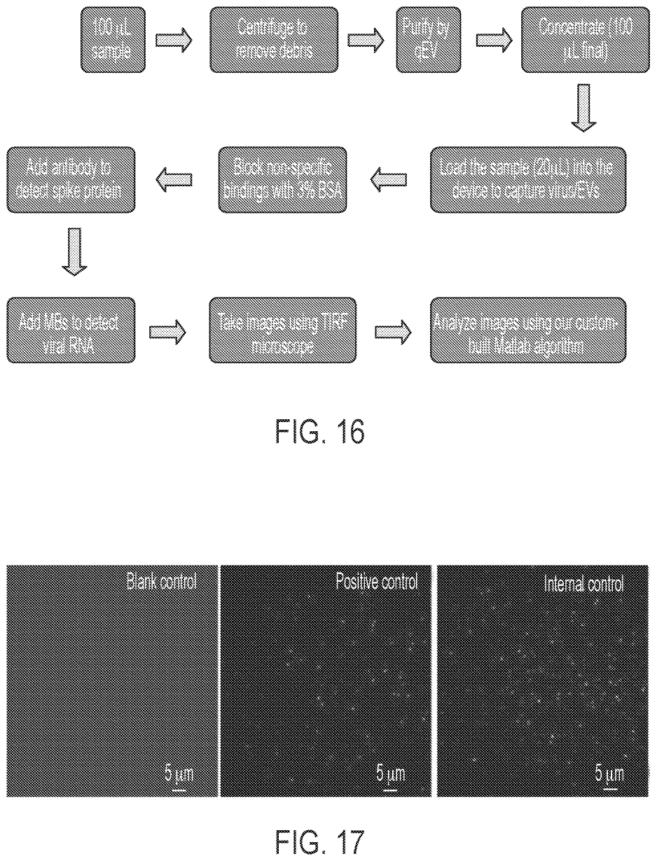

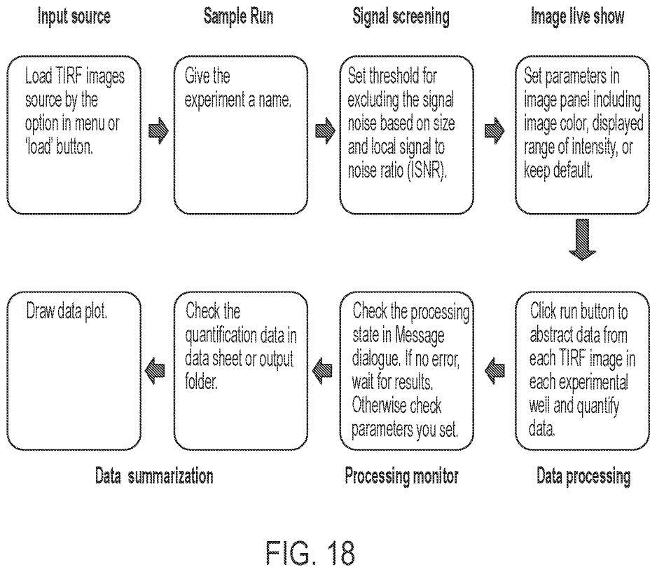

Virion-RNA detection in plasma-derived EVs of post-acute sequelae of COVID-19 (PASC) patients. Patients with COVID-19 may suffer a heterogeneous set of symptoms post-infection, which is referred to as PASC, ranging from neurologic to cardiovascular symptoms, and affecting various organs. Therefore, virion-RNA is present in EVs of PASC patients originating from infected tissue. PASC patients were recruited as patients with ongoing, relapsing, or new symptoms persisting beyond 30 days of acute infection. Samples from seven patients with PASC were used in the investigation (Table 8-9), whereby their plasma was collected serially at three time points and co-detected with qRT-PCR and the BARA. Tunable resistive pulse sensing (TRPS) on the size distribution of EVs from saliva and plasma revealed similar profiles to SARS-CoV-2 virions (FIG. 13A-B). Therefore, it was necessary to specifically isolate EVs from SARS-CoV-2 virions. The use of an antibody cocktail targeting CD63 and CD9, which are tetraspanins enriched in various subpopulations of EVs, revealed an absence of signal in a patient saliva sample with COVID-19, but an enrichment of CD63+ single EVs (FIG. 14). On the other hand, capturing particles with antibodies targeting the S1 and S2 subunits of the spike glycoprotein revealed a loss of CD63+ single EVs (FIG. 14). Furthermore, various isolation methods, including dextran-based precipitation, size-exclusion chromatography, and thrombin for the cleavage of fibrinogen were conducted to retrieve EVs from plasma. The EVs isolated with thrombin produced CD63 and CD81 signals at higher frequencies compared to the other isolation methods (FIG. 15A-B), which had less loss of EVs than the other protocols (FIG. 15C).

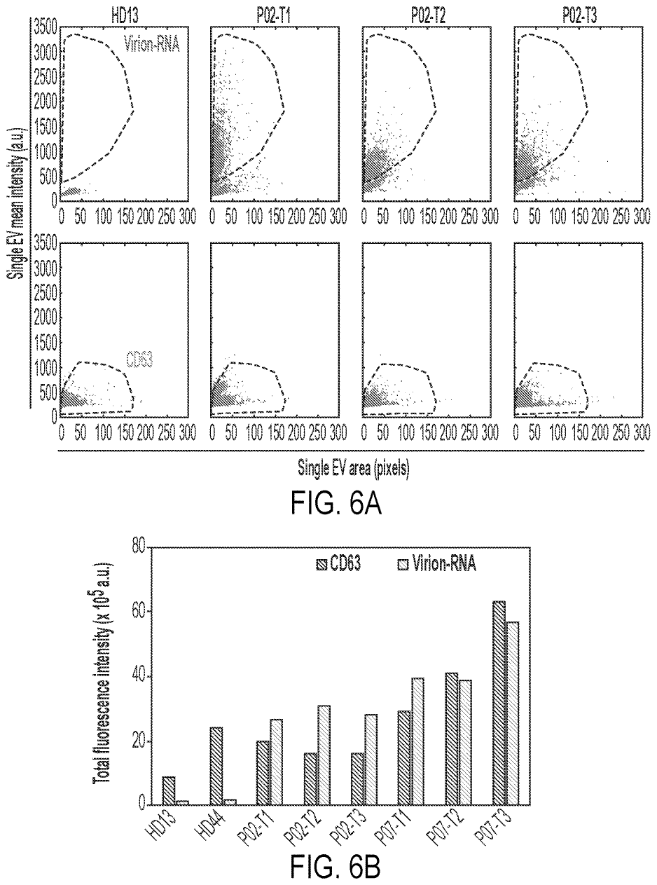

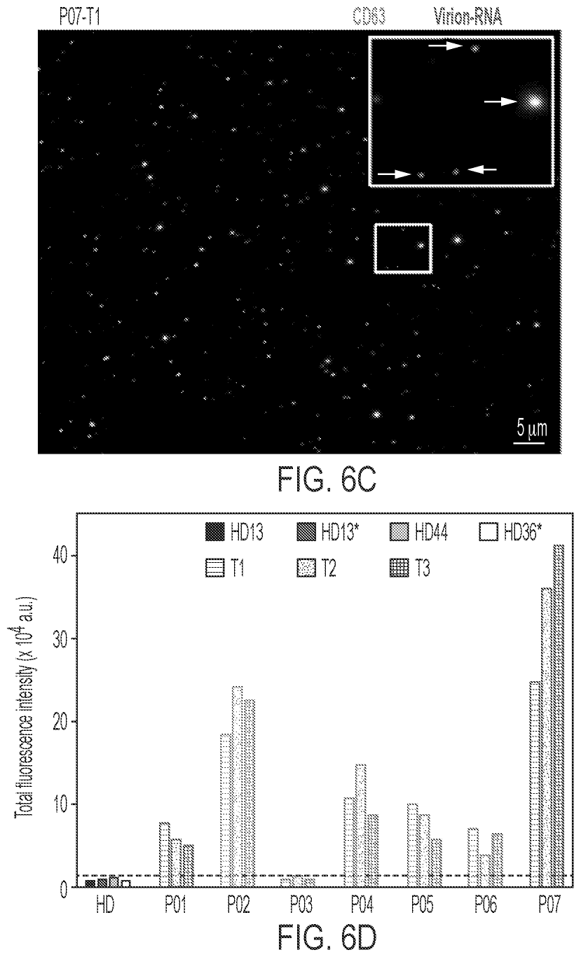

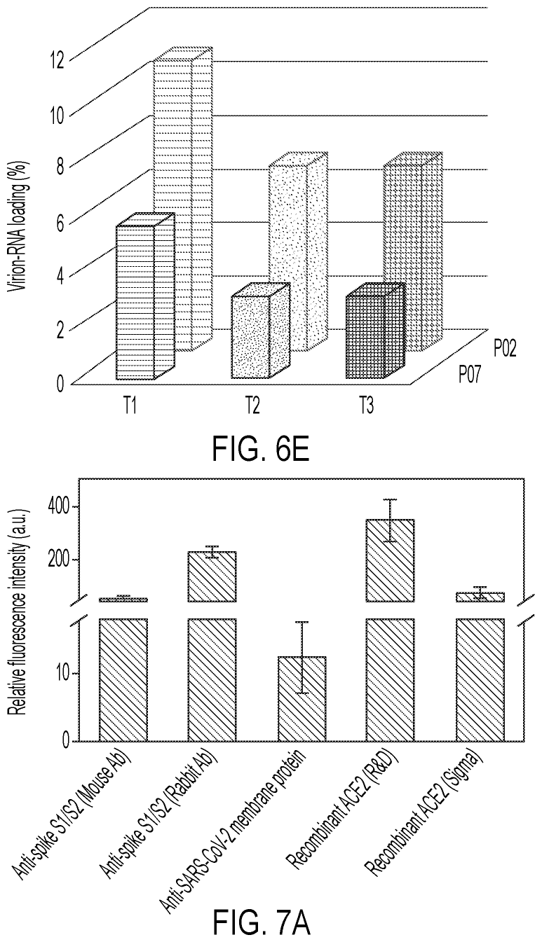

To test the presence of virion-RNA in IHD-EVs, thrombin-treated plasma from PASC patients was screened with the BARA whereby EVs were isolated via positive immunoselection with CD63/CD9-targeting antibodies and detected for the nucleocapsid-encoding RNA. A high-intensity subpopulation for nucleocapsid-encoding RNA expression was detected in single IHD-EVs after long timeframes extending past 200 days, which was absent in healthy donor serum, while CD63 expression remained the same in single EVs across healthy donor and PASC patient samples (FIG. 6A). Total expression of CD63 and the nucleocapsid-encoding RNA demonstrated that the detection of IHD-EVs was EV-independent, as CD63 signals remained high for healthy donors and PASC patients (Student's two-tailed t-test, p=0.35), whereas nucleocapsid-encoding RNA signals were higher for PASC patients (FIG. 6B; Student's two-tailed t-test, p=0.0055). Furthermore, the presence of CD63+IHD-EVs containing nucleocapsid-encoding RNA was demonstrated by their colocalization on single IHD-EVs (FIG. 6C). Interestingly, colocalization analysis with the BARA revealed that the loading percentage of nucleocapsid-encoding RNA in IHD-EVs decreased over the course of infection (FIG. 6E). Although qRT-PCR and the BARA did not correlate in levels of expression for the nucleocapsid-encoding RNA in IHD-EVs (Pearson's correlation coefficient, r=0.08), likely due to qRT-PCR being at the cusp of its LoD (Table 8), the qRT-PCR demonstrated positivity whereby the BARA provided more holistic profiles of the nucleocapsid-encoding RNA levels as a function of time (FIG. 6D). Therefore, the BARA demonstrated the packaging of virion-RNA in IHD-EVs of patients with symptoms post-infection, possibly describing residual virion-RNA as the culprit for the long-term symptoms associated with PASC.

### DISCUSSION

The BARA demonstrates the translation of single-EV technologies for clinical application. Progressing toward single-EV detection has afforded unprecedented sensitivities surpassing enzyme-linked immunosorbent assay (ELISA) for protein detection and qRT-PCR for microRNA and messenger RNA detection. Herein, the BARA outperformed qRT-PCR by detecting the spike glycoprotein and nucleocapsid-encoding RNA in single SARS-CoV-2 virions. The enhanced sensitivity allowed for the detection of a subpopulation of COVID-19 patients that often eluded qRT-PCR tests, which were asymptomatic and were partly responsible for the vast undetectable spread of the SARS-CoV-2 virion. Another subpopulation of COVID-19 patients that were partly responsible for the spread of the disease was recently infected individuals who often presented false-negative results. Detecting patients upon infection before replication whereby virion levels are too low for contagion is a diagnostic window to limit the spread of virion-mediated outbreaks by enforcing a bottleneck to virion transmission, such as is observed for highly mortal pathogenic diseases like Ebola. Therefore, single-virion methods aid in the rapid detection and subsequent mitigation of outbreaks via efficiently detecting patients with low-virion counts.

Another benefit to utilizing single-EV methods is the facile deconvolution of vesicular heterogeneity via in situ colocalization of biomolecular signals. The BARA offered a unique qualitative perspective through the colocalization of biomolecular signals, allowing the visualization of mutation-harboring virions and virion-RNA within IHD-EVs in PASC patients. Due to the growing risk of zoonoses, an active field in emerging infective diseases is the identification of mutations that may lead to animal-to-human transmission or vice versa. The BARA can aid in identifying rates of mutation and the likelihood of zoonoses via the serial colocalization of human-infecting genes with a housekeeping gene in various animals that pose a threat to interspecies spillover. On the other hand BARA determined that PASC patients contain virion-RNA in IHD-EVs after extended timeframes. Given the heterogeneous symptoms experienced by PASC patients, colocalizing organ-specific biomarkers with EV biomarkers and virion-RNA elucidate the causations of symptoms or even predict symptoms before their onset. Moreover, antibodies screened in high throughput via IF on various virion strains determine the efficacy of vaccine targets.

Lastly, the tunability of the BARA provides a customizable framework for many virion-mediated diseases. Herein, demonstrated is the ability of the BARA to detect antigens and nucleic acids simultaneously in single Influenza A and RSV virions by further designing molecular beacons and fluorescent-dye-conjugated antibodies tailored to the respective biomolecules. Apart from providing the unique multifaceted detection of single virions, the combination of IF and FISH for detecting antigens and nucleic acids in single virions enhanced the sensitivity of the BARA compared to single biomolecule detection. The increased sensitivities afforded the detection of single SARS-CoV-2 virions in complex biofluids and the sensitive identification of COVID-19 patients insofar as sensitivities of 100% in the case of saliva samples were observed.

### Materials and Methods

The BARA fabrication. High-precision, 24×75×0.15 mm, borosilicate glass coverslips (D 263® M; Schott AG, Mainz, Germany) were cleaned with ethanol followed by deionized (DI) water in an ultrasonic bath for 5 min each. After repeating the cleaning process, the coverslips were dried with nitrogen gas. The coverslips were cleaned with a UV-ozone cleaner (Jelight, Irvine, CA) for 15 min. A 2-nm film of titanium was first deposited onto the cleaned coverslips via electron beam evaporation (DV-502A; Denton Vacuum, Moorestown, NJ) to facilitate the adhesion of gold to the surface. Utilizing the same technique, a 10-nm film of gold was deposited atop the titanium layer. The gold-coated coverslips were then submerged into a solution of thiolated molecules to functionalize the gold with biotin motifs, which was comprised of β-mercaptoethanol (BME; Sigma-Aldrich, St. Louis, MO), 2 kDa methoxy-poly(ethylene glycol)-thiol (mPEG-SH; Laysan Bio, Arab, AL), and 2 kDa biotin-PEG-SH (Nanocs, New York, NY) at a molar ratio of 95:3:2, respectively, in 200 proof ethanol (Thermo Fisher Scientific, Waltham, MA). The coverslips were incubated in the solution overnight at room temperature in a dark environment. Excess thiolated molecules were rinsed away with ethanol. The biotin-functionalized, gold-coated coverslips (referred to as biochips) were then dried with nitrogen gas and fastened to a 64-well ProPlate® microarray system (Sigma-Aldrich, St. Louis, MO). Antibody functionalization of the biochip surface. The working volume utilized for an individual well was 20 μL, which was kept constant for the different solutions added into the wells. Furthermore, all incubation steps were performed on a shaker to ensure a uniform coating of the solution throughout the well surface. Before antibody functionalization, each well was washed by pipetting DI water up and down 10 times. Then, a 50-μg/mL solution of NeutrAvidin (NA; Thermo Fisher Scientific) diluted in PBS (Thermo Fisher Scientific) was added into each well and incubated at room temperature for 1 hr to bind to the biotin motifs functionalized on the gold surface of the biochip. Excess NA was rinsed away by pipetting PBS up and down 10 times. The rinsing process was repeated three times. Capture antibodies and recombinant proteins previously biotinylated via the EZ-Link micro Sulfo-NHS-biotinylation kit (Thermo Fisher Scientific) were diluted to a concentration of 10 μg/mL in a 1% (w/v) solution of bovine serum albumin (BSA; Sigma-Aldrich) in PBS. Specific capture antibodies or recombinant proteins to immobilize subpopulations (Table 1) were added into each well and incubated at room temperature for 1 hr. Excess antibodies and recombinant proteins were rinsed away by pipetting PBS up and down 10 times for three repetitions.

Saliva collection. Following Institutional Review Board protocol 2021H0246 (Biomedical Sciences Committee at The Ohio State University), a de-identified cohort comprising 30 healthy donors, 33 symptomatic patients, and 20 asymptomatic patients was enrolled from which saliva samples were collected. Pooled healthy donor saliva was defined as the combined saliva from five healthy donors. After collection, the saliva was centrifuged at 2000×g for 10 min and stored at −80° C. All saliva samples were inactivated at 56° C. for 30 min prior to purification.

Nasopharyngeal swab (NS) collection. A de-identified cohort of 19 healthy donors and 40 patients was collected following the Institutional Review Board protocol 2021H0246 (Biomedical Sciences Committee at The Ohio State University). All NS samples were inactivated at 56° C. for 30 min prior to purification.

Plasma collection. Plasma samples were provided from a cohort of 7 COVID-19 patient participants with written informed consent, in accordance with the Code of Federal Regulations Title 45: Public Welfare Part 46: Protection of Human Subjects (45 CFR 46). After collection, the plasma samples were stored at −80° C. All plasma samples were inactivated at 56° C. for 30 min prior to purification. Defibrination of plasma samples was performed by adding 4.4 U/mL of thrombin (TMEXO-1, System Biosciences) to the plasma samples, incubating for 5 min, centrifuging at 10000× g at room temperature for 5 min, and collecting the supernatant.

Microorganism collection. Viruses, bacteria, and fungi were obtained from the Biodefense and Emerging Infections (BEI) Resources Repository, American Type Culture Collection (ATCC), and the Department of Pathology at The Ohio State University Wexner Medical Center. All viruses, bacteria, and fungi were inactivated at 56° C. for 30 min and diluted to the tested concentrations (Table 2).

Biofluid sample purification. Biofluid samples were purified via size-exclusion chromatography (qEV; Izon Science, Christchurch, New Zealand) according to the manufacturer's instructions. Briefly, a 200 μL biofluid sample was introduced through the column pre-wetted with PBS, whereby fractions 7-12 were collected. The purified samples were then re-concentrated to 200 μL with spin columns (10 kDa MWCO, Millipore Sigma Amicon Ultra Centrifugal Filter Unit, Fisher Scientific) at 4° C. at 3000× g.

Molecular beacon hybridization to membrane-enveloped virion RNA. Molecular beacons (Table 10) were diluted to a concentration of 5 μM in 12.5×Tris EDTA (TE) buffer (Sigma-Aldrich) diluted in DI water to stabilize the molecular beacons and permeabilize the membrane encasing the RNAs. The molecular beacon cocktail was diluted 25 times within the purified biofluid sample and allowed to incubate for 2 hr at 37° C. in a dark environment to facilitate molecular beacon hybridization to the target RNA.

Capture of virions and extracellular vesicles (EVs). A 3% (w/v) solution of BSA in PBS was incubated in each well at room temperature for 1 hr to block non-specific particle capture. After the removal of BSA, the biofluid samples containing virions or EVs (including pre-hybridized and untreated samples) were subsequently incubated in the wells of the BARA for 2 hr at room temperature in a dark environment. For the untreated samples, excess virions and EVs were washed by pipetting PBS up and down 10 times for a total of 3 repetitions. For the pre-hybridized samples, PBS was incubated in the wells for 5 min then pipetted up and down 10 times to remove excess virions and EVs and unhybridized molecular beacons. The rinsing step was repeated 4 times in a dark environment.

Immunofluorescence of membrane proteins. A 3% (w/v) solution of BSA in PBS was incubated in each well at room temperature for 1 hr to block the non-specific adhesion of the fluorescent-dye-conjugated antibodies. After withdrawing the BSA solution, a 1-μg/mL solution of the fluorescent-dye-conjugated antibodies (Table 1) in 10% (w/v) normal goat serum (NGS; Thermo Fisher Scientific) was subsequently incubated in the wells for 1 hr at room temperature in a dark environment. Excess fluorescent-dye-conjugated antibodies were rinsed and removed by incubating in PBS for 5 min then pipetting up and down the solution 10 times. The rinsing step was repeated 3 times in a dark environment.

Image acquisition and processing. A 10×10 array of images was acquired via TIRFM (Nikon, Melville, NY) for each well with a 100× objective and immersion oil to reduce surface refraction. Exposure times and laser power were maintained across experiments to ensure the consistency of the assay. TIRFM images were quantified by measuring the total and mean fluorescence intensity of each spot detected by TIRFM. Histograms were generated with the total and mean intensities of the single spots detected by TIRFM. Scatter plots were generated with the mean intensity and size of the single spots detected by TIRFM. Relative and total fluorescent intensities of the sample were obtained from custom-built algorithms that were previously reported.

Real-time quantitative reverse transcription-polymerase chain reaction (qRT-PCR). Membrane-enveloped RNA was isolated with the miRNeasy Serum/Plasma kit (Qiagen, Hilden, Germany) and the Single Cell RNA Purification Kit (Sigma-Aldrich) according to the manufacturer's instructions. The isolated RNA was combined with random primers (Thermo Fisher Scientific) and was heated to 70° C. for 2 min to ensure that the target RNA was single-stranded and cooled to 4° C. to anneal the primers. A solution containing Moloney murine leukemia virus reverse transcriptase (MMLV-RT; Thermo Fisher Scientific) and deoxyribonucleotide triphosphate (dNTP; Thermo Fisher Scientific) in a buffer comprised of dithiothreitol (DTT; Thermo Fisher Scientific), RNaseOUT™ Recombinant Ribonuclease Inhibitor (Thermo Fisher Scientific), and Maxima First Strand cDNA Synthesis Kit Reaction Mix (Thermo Fisher Scientific) was heated to 42° C. for 1 hr to synthesize cDNA, then heated to 95° C. for 5 min to deactivate the RT, and subsequently cooled to 4° C. for storage purposes. The cDNA was introduced to a TaqMan™ Fast Advanced Master Mix (Thermo Fisher Scientific) and combined with probes targeting the open reading frame (ORFlab), the spike protein, the nucleocapsid protein, and the human ribonuclease P RNA component H1 gene (RPPH1; RNase P) as a positive control as provided by the TaqMan™ 2019-nCoV Assay Kit vl (Thermo Fisher Scientific). Real-time qRT-PCR was performed on the sample with an activation step of 95° C. for 20 s followed by 45 cycles of denaturing at 95° C. for 3 s and annealing and extending at 60° C. for 30 s.

Transmission electron microscopy (TEM). Two 20 μL droplets of water for injection (WFI) and two 20 μL droplets of negative stain (UranyLess EM stain, Electron Microscopy Sciences) were placed on a strip of parafilm. The TEM grid was subjected to plasma treatment for 1 min. Then, 10 μL of a SARS-CoV-2 virion solution was carefully placed onto the treated surface of the grid. The virions solution was incubated on the surface of the grid surface for 1 min and gently blotted with filter paper to remove excess liquid. The virion-coated grids were submerged into the WFI droplet and blotted dry using filter paper. The process was repeated with the second WFI droplet. The virion-coated grids were stained via submersion into the first droplet of negative stain, followed by blotting, and then submerging again into the second droplet of the negative stain. The grid was allowed to incubate in the stain for approximately 22 seconds before gently wicking away the excess solution using filter paper. To ensure thorough drying, the stained grids were stored in a grid box overnight. Afterward, TEM imaging was performed using a Tecnai TF-20 microscope (FEI Company, Hillsboro, OR) operating at 200 kV.

Scanning electron microscopy (SEM). The BARA with SARS-CoV-2 virions captured on the surface with recombinant ACE2 was dehydrated with increasing ethanol concentrations (70, 85, 95, and 100% (v/v)) for 5 min each. Lastly, the BARA was immersed in hexamethyldisilazane (HMDS, Sigma-Aldrich) for 10 min and air-dried overnight. The samples were imaged using an Apreo 2 SEM (FEI Company, Hillsboro, OR).

Flow cytometry. SARS-CoV-2 virions were stained with antibodies targeting the spike glycoprotein and molecular beacons targeting the nucleocapsid-encoding RNA (Table 10), according to the previous strategies. MLV virions and PBS were utilized as a negative control and were stained following the same protocol. The samples were then imaged using ImageStream® X mark II (MilliporeSigma, Burlington, MA, USA).

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES
1.
S Gene
SEQ ID NO: 1
+GCT T+C /iFluorT/ +GCT +AAT +CTT +GCT GCT AGC AAG ATT AGC /3BHQ_1/
2.
S Gene
SEQ ID NO: 2
+GCA +ACA +CA+G/iFluorT/T+G CT+G ATT CTC AAT CAG CAA CTG /3BHQ_1/
3.
N Gene
SEQ ID NO: 3
[+C]GG[+G]TG[+C]CA[+A]TG[+T]GA[+T] CTTTTGAGATCACATTGG
4.
N Gene
SEQ ID NO: 4
[+C]CA[+T]TG[+C]CA[+G]CC[+A]TT[+C] TAGCTAGAATGGCTGG
5.
N Gene
SEQ ID NO: 5
[+G]TT[+G]AG[+T]GA[+G]AG[+C]GG[+T]GA ACA CCG CTC TCA
6.
Delta
SEQ ID NO : 6
[+C] CT[+C]AC[+T]T[+T] C[+C]A T[+C]C[+A]AC TTT TGG ATG GAAA
7.
Delta
SEQ ID NO : 7
[+C] GC[+C]GA[+C]GA[+G] AA[+T]TA[+G]TCTGAGTCGACTAATTCTCG
8.
Gene-N2 (NC_045512.2) Location 29190-29210
SEQ ID NO: 8
+CTG +AAG /iCy3/ +CGC +TGG +GGG +CAA ATT CCC CCA GCG /3BHQ_2/
1.
Gene-N4 (NC_045512.2) Location 28699-28719
SEQ ID NO: 9
+CGG +GTG /iCy3/+CCA +ATG +TGA +TCT TTT GAG ATC ACA TTG G/3BHQ_2/
10.
Gene-N7 (NC_045512.2) Location 28531-28551
SEQ ID NO: 10
+CGT +CTG /iCy3/+GT A +GCT +CTT+CGG TAG CGA AGA GCT AC/3BHQ_2/
11.
Gene-delta F157 (B.1 .617.2) (OX014251.1) Location 22011-22028
SEQ ID NO: 11
[Cy3][+C]CT[+C]AC[+T]T[+T] C[+C]AT[+C]C[+A]AC TTT TGG ATG GAAA[BHQ2]
12.
Gene L452R (B.1 .617.2) (OX014251.1) Location 22892-22917
SEQ ID NO: 12
[Cy3][+C]T[+A] T[+A]C[+C]G[+G] T[+A]A[+T]TA[+T]AA TTA CCA CCAATT ATA ATT
ACC G[BHQ2]
13.
Gene delta H69 (BA.1) (OR624070.1) Location 21682-21703
SEQ ID NO: 13
+CCA +GAT /iCy3/+ATA +GCA +TGG+AAC CAA GTT CCA TGC TAT /3BHQ_2/
14.
Gene Influenza A (H1N1,NP) (OR_637833.1) Location 616-637
SEQ ID NO: 14
+GTC +CAT /iCy3/+TTT +CAC +CCC+TCC AGA AGA GGG GTG AAA /3BHQ_2/
15.
Gene RSV (OQ_848527.1) Location 5944-5966
SEQ ID NO: 15
+CTG +GCT /iCy3/+CGA +TTG +TTT+GTT GCT TGA ACA AAC AA T CG /3BHQ_2/
16.
SEQ ID NO: 16
AATTTGCCCCCAGCGCTTCAG
17.
SEQ ID NO: 17
AAAAGATCACATTGGCACCCG
18.
SEQ ID NO: 18
CTACCGAAGAGCTACCAGACG
19.
SEQ ID NO: 19
AAAGTTGGATGGAAAGTG
20.
SEQ ID NO: 20
GGTGGTAATTATAATTACCGGTATAG
21.
SEQ ID NO: 21
CTTGGTTCCATGCTATATCTGG
22.
SEQ ID NO: 22
TTCTGGAGGGGTGAAAATGGAC
23.
SEQ ID NO: 23
CAAGCAACAAACAATCGAGCCAG

The “+” or the “[+]” indicates a locked nucleic acid (LNA) nucleotide.

### Tables

Table 1 depicts a list of antibodies and proteins used for virion and EV capture and detection.

| Antibody/Protein | | | Catalog no. | Supplier |
|---|---|---|---|---|
| Capture | SARS-CoV-2 | RecombinantACE2 | BT933 | R&D Systems |
| | | RecombinantACE2 | SAE0064 | Sigma-Aldrich |
| | | Anti-SARS-COV-2 membrane protein | NBP3-05698 | Novus Biologicals |
| | | Anti-spike S1 (Mouse MAb) | MAB105403 | R&D Systems |
| | | Anti-spike S2 (Mouse MAb) | MAB10557 | R&D Systems |
| | | Anti-spike S1 (Rabbit Mab) | 40150-R007 | Sino Biological |
| | | Anfi-spike S2 (Rabbit Pab) | 40590-T82 | Sino Biological |
| | EVs | Anti-CD63 | MAB5048 | R&D Systems |
| | | Anti-CD9 | MAB1880 | R&D Systems |
| | RSV | Anti-respiratory syncytial virus | AB19986 | Abcam |
| | Influenza A | Anti-influenza A | AB20841 | Abcam |
| Detection | SARS-CoV-2 | Anti-SARS-CoV-2 spike protein | 51-6491-82 | Invitrogen |
| | EVs | Anti-CD63 (Alexa Fluor ® 488) | SC-5275 AF488 | Santa Cruz |
| | | | | Biotechnology |
| | RSV | Anti-respiratory syncytial virus (FITC) | AB20391 | Abcam |
| | Influenza A | Anti-influenza A (FITC) | AB20388 | Abcam |

Table 2 depicts testing concentrations for potential cross-reactive microorganisms.

| potential cross reactant | | concentration of saliva matrix |
|---|---|---|
| Virus | Human oronavirus 229B | 1.00E+06 TCID₅₀/mL |
| | Human coronavirus OC43 | 1.00E+06 TCID₅₀/mL |
| | Hunan coronavirus NL63 | 1.00E+06 TCID₅₀/mL |
| | Human Metapheumovirus (hMPB | 1.00E+06 TCID₅₀/mL |
| | Parainfluenza virus 1 | 1.60E+06 TCID₅₀/mL |
| | Parainfluenza virus 2 | 1.00E+06 TCID₅₀/mL |
| | Parainfluenza virus 3 | 1.00E+06 TCID₅₀/mL |
| | Parainfluenza virus 4A | 1.60E+04 TCID₅₀/mL |
| | Parainfluenza virus 4B | 1.00E+06 TCID₅₀/mL |
| | Influenza A | 5.20E+06 TCID₅₀/mL |
| | Influenza B | 1.00E+06 TCID₅₀/mL |
| | Enterovirus | 1.00E+06 TCID₅₀/mL |
| | Rhinovirus | 8.00E+04 TCID₅₀/mL |
| | Cytomegalovirus (CMV) | 1.00E+06 TCID₅₀/mL |
| Bacteria | Haemophilus influenza | 1.00E+06 CFU/mL |
| | Streptococcus pneumoniae | 1.00E+06 CFU/mL |
| | Streptococcus pyogenes | 1.00E+06 CFU/mL |
| | Bordetella pertussis | 1.00E+06 CFU/mL |
| | Mycoplasma salivarium | 1.00E+06 CFU/mL |
| | Chlamydia pneumoniae | 1.00E+06 CFU/mL |
| | Legionella pneumoniae | 1.00E+06 CFU/mL |
| | Staphylococcus aureus | 1.00E+06 CFU/mL. |
| | Porphyromonas gingivalis | 1.00E+06 CFU/mL |
| | Streptococcus mitis | 1.00E+06 CFU/mL |
| | Pseudomonas aeruginosa | 1.00E+06 CFU/mL |
| | Streptococcus salivarius | 1.00E+06 CFU/mL |
| | Streptococcus mutans | 1.00E+06 CFU/mL |
| | Moraxella catarrhalis | 1.00E+06 CFU/mL |
| | Nocardia sp. | 1.00E+06 CFU/mL |
| Fungi | Candida albicans | 1.00E+06 CFU/mL |

Table 3 depicts testing concentrations for potential endogenous and exogenous substances.

| interference substance | concentration of saliva matrix | |
|---|---|---|
| Chloraseptic Menthol (Orajel) | 1.5 | mg/ml |
| CVS Nasal drops (Phenylephrine) | 15% | v/v |
| Zicam | 5% | v/v |
| Homeopathic (Alkalol) | 1:10 | dilution |
| Naso GEL (NeilMed) | 5% | v/v |
| Afrin (Oxymetazoline) | 15% | v/v |
| CVS Nasal Spray (Cromolyn) | 15% | v/v |
| Chioroseptic Sore Throat spray (Phenol) | 5% | v/v |
| Fluticasone Propionate | 5% | v/v |
| Robitussin | 5% | v/v |
| Act dry mouth lozenges (xylitol) | 3 | mg/ml |
| Nyquil (Acetaminophen, Doxylamine succinate, Dextromethorphan HBr) | 5% | v/v |
| Human Genomic DNA | 10 | ng/μl |
| Mucin: bovine submaxillary gland, type IS | 2.5 | μg/ml |
| Vaseine (Petroleum Jelly) | 0.25 | mg/ml |
| Nicotine | 0.03 | mg/ml |
| Tobramycin | 4 | μg/ml |
| Mupirocin | 10 | mg/ml |
| Tamiflu | 5 | mg/ml |

Table 4 depicts accelerated stability test for the BARA at three times the limit of detection (LoD).

| 10° C. | | 20° C. | | 25° C. | | 30° C. | |
|---|---|---|---|---|---|---|---|
| Day | Response | Day | Response | Day | Response | Day | Response |
| 0 | 1.00 | 0 | 1.00 | 0 | 1.00 | 0 | 1.00 |
| 9 | 1.02 | 10 | 1.01 | 10 | 1.00 | 10 | 0.99 |
| 15 | 1.01 | 15 | 0.86 | 15 | 0.99 | 15 | 0.98 |
| 20 | 0.81 | 21 | 0.83 | 20 | 0.97 | 20 | 0.92 |
| 23 | 0.85 | 25 | 0.83 | 24 | 0.89 | 24 | 0.87 |
| 30 | 0.84 | 30 | 0.79 | 30 | 0.84 | 30 | 0.79 |
| 35 | 0.88 | 32 | 0.79 | 35 | 0.69 | 35 | 0.57 |
| 37 | 0.78 | 36 | 0.78 | 38 | 0.57 | 38 | 0.27 |
| 41 | 0.94 | 39 | 0.68 | 41 | 0.33 | 41 | 0.28 |
| 47 | 0.86 | 45 | 0.61 | 45 | 0.16 | 45 | 0.07 |

Table 5 depicts degradation rate constants (kj) at different temperatures (Tj).

| Temp (Tj) | Rate Constant (kj) |
|---|---|
| 10° C. | 0.0040 |
| 20° C. | 0.0075 |
| 25° C. | 0.0118 |
| 30° C. | 0.0177 |

Table 6 depicts detailed information on the patients and healthy donors enrolled for saliva collection.

| Sample ID | N genes Ct value |
|---|---|
| Healthy HD001 | N.A. |
| Healthy HO002 | N.A. |
| Healthy HO004 | N.A. |
| Healthy HD005 | N.A |
| Healthy HD006 | N.A. |
| Healthy HD007 | N.A. |
| Healthy HD009 | N.A |
| Healthy HD010 | N.A |
| Healthy HO011 | N.A. |
| Healthy HD012 | N.A. |
| Healthy HD013 | N.A. |
| Healthy HD014 | N.A. |
| Healthy HD015 | N.A. |
| Healthy HD016 | N.A. |
| Healthy HD017 | N.A |
| Healthy HO018 | N.A. |
| Healthy HO019 | N.A. |
| Healthy HD020 | N.A. |
| Healthy HD022 | N.A. |
| Healthy HD023 | N.A. |
| Healthy HD024 | N.A. |
| Healthy HD025 | N.A |
| Healthy HO026 | N.A. |
| Healthy HO027 | N.A. |
| Healthy HO028 | N.A. |
| Healthy HD031 | N.A. |
| Healthy HD032 | N.A. |
| Healthy HD033 | N.A. |
| Healthy HD034 | N.A |
| Healthy HO035 | N.A |
| Patient 150 | 18.9 |
| Patient 68 | 24.1 |
| Patient 162 | 25.9 |
| Patient 112 | 26.2 |
| Patient 7 | 27.4 |
| Patient 22 | 27.5 |
| Patient 70 | 28.1 |
| Patient 148 | 28.2 |
| Patient 72 | 28.3 |
| Patient 31 | 28.3 |
| Patient 60 | 28.4 |
| Patient 165 | 29.2 |
| Patient 114 | 29.8 |
| Patient 23 | 30.8 |
| Patient 56 | 31.5 |
| Patient 71 | 31.5 |
| Patient 61 | 31.8 |
| Patient 24 | 33.7 |
| Patient 53 | 33.9 |
| Patient 32 | 34.4 |
| Patient 52 | 35.1 |
| Patient 106 | 24.5 |
| Patient 54 | 24.9 |
| Patient 89 | 29.7 |
| Patient 51 | 34.4 |
| Palieni 62 | 35.2 |
| Patient 26 | 35.3 |
| Patient 33 | 35.9 |
| Patient 27 | 36.1 |
| Patient 37 | 36.2 |
| Patient 25 | 36.3 |
| Patient C4 | 39.0 |
| Patient C5 | 39.0 |
| P401 | 20.00 |
| P402 | 18.90 |
| P403 | 23.10 |
| P404 | 21.30 |
| P405 | 21.90 |
| P406 | 19.00 |
| P407 | 25.10 |
| P408 | 23.40 |
| P409 | 25.10 |
| P410 | 27.20 |
| P411 | 24.50 |
| P412 | 26.50 |
| P413 | 26.70 |
| P414 | 28.00 |
| P415 | 28.10 |
| P416 | 26.30 |
| P417 | 28.80 |
| P418 | 27.90 |
| P419 | 27.40 |
| P420 | 27.60 |

Table 7 depicts detailed information on the patients and healthy donors enrolled for NS collection.

| Sample ID | N genes Ct value |
|---|---|
| Healthy H4891 | N.A. |
| Healthy H4892 | N.A. |
| Healthy H4893 | N.A. |
| Healthy H4894 | N.A. |
| Healthy H4895 | N.A. |
| Healthy H4896 | N.A |
| Healthy H4900 | N.A. |
| Healthy H4902 | N.A |
| Healthy H4904 | N.A. |
| Healthy H4905 | N.A. |
| Healthy H4907 | N.A. |
| Healthy H4910 | N.A. |
| Healthy H4911 | N.A. |
| Healthy H4912 | N.A. |
| Healthy H4913 | N.A. |
| Healthy H4916 | N.A. |
| Healthy H4917 | N.A. |
| Healthy H4918 | N.A. |
| Healthy H4926 | N.A. |
| Patient 4013 | 12.9 |
| Patient 3968 | 13.0 |
| Patient 3975 | 13.3 |
| Patient 4024 | 13.6 |
| Patient 4015 | 13.6 |
| Patient 3971 | 13.7 |
| Patient 3979 | 13.8 |
| Patient 4014 | 13.8 |
| Patient 4047 | 14.3 |
| Patient 4002 | 14.4 |
| Patient 3972 | 14.5 |
| Patient 4036 | 14.7 |
| Patient 338 | 20.4 |
| Patient 136 | 21.2 |
| Patient 134 | 21.3 |
| Patient 166 | 21.3 |
| Pation: 137 | 21.6 |
| Patient 046 | 21.6 |
| Patient 287 | 21.7 |
| Patient 60 | 21.9 |
| Patient 171 | 22.4 |
| Patient 4028 | 22.4 |
| Patient 3994 | 22.8 |
| Patient 102 | 22.8 |
| Patient 018 | 23.2 |
| Patient 4034 | 23.2 |
| Patient 47 | 23.3 |
| Patient 59 | 23.9 |
| Patient 174 | 24.6 |
| Patient 168 | 24.8 |
| Patient 071 | 25.4 |
| Patient 69 | 25.7 |
| Patient 054 | 26.2 |
| Patient 053 | 26.6 |
| Patient 65 | 27.0 |
| Patient 110 | 27.4 |
| Patient 120 | 27.5 |
| Patient 46 | 28.0 |
| Patient 38 | 28.1 |
| Patient 163 | 28.2 |

Table 8 depicts detailed information on the patients enrolled for the post-acute sequelae SARS-CoV-2 infection (PASC) study.

| | Viral N | Human | Observation days | Onset of symptoms | Symptom | WHO |
|---|---|---|---|---|---|---|
| Sample ID | gene Ct | RNase P Ct | since enrollment | before enrollment | onset | ordinal scale |
| P01-T1 | 36.91 | 29.40 | 4.54 | 6.17 | 10.71 | 5 |
| P01-T2 | 38.08 | 29.83 | 6.58 | 6.17 | 12.75 | 3 |
| P01-T3 | 34.52 | 33.49 | 247.54 | 6.17 | 253.71 | 3 |
| P02-T1 | 37.08 | 32.24 | 0.04 | 8.46 | 8.5 | 4 |
| P02-T2 | 35.59 | 33.06 | 2.79 | 8.46 | 11.25 | 3 |
| P02-T3 | 34.84 | 36.81 | 198.83 | 8.46 | 207.26 | N.A. |
| P03-T1 | 36.96 | 33.12 | 1 | 2.75 | 3.75 | 3 |
| P03-T2 | 35.18 | 31.11 | 3.96 | 2.75 | 6.71 | 4 |
| P03-T3 | 33.31 | 33.68 | N.A. | N.A. | N.A. | N.A. |
| P04-T1 | 34.66 | 24.80 | 0.83 | 3 | 3.83 | 6 |
| P04-T2 | 36.56 | 33.23 | 11.75 | 3 | 14.75 | 3 |
| P04-T3 | 33.63 | 33.13 | 135.08 | 3 | 138.08 | N.A. |
| P05-T1 | 36.13 | 30.66 | 2.54 | 7.25 | 9.79 | 4 |
| P05-T2 | 36.41 | 34.01 | 14.58 | 7.25 | 21.83 | 1 or 2 |
| P05-T3 | 34.62 | 34.22 | 102.58 | 7.25 | 109.83 | <=2 |
| P06-T1 | 35.17 | 29.67 | 0.67 | 8.21 | 8.88 | 6 |
| P06-T2 | 37.18 | 28.18 | 4.83 | 8.21 | 13.04 | 6 |
| P06-T3 | 34.81 | 33.49 | 106.83 | 8.21 | 115.04 | <=2 |
| P07-T1 | 36.97 | 28.93 | −0.5 | 4.71 | 4.21 | 4 |
| P07-T2 | 36.70 | 29.94 | 4.71 | 4.71 | 9.42 | 1 or 2 |
| P07-T3 | 34.64 | 34.43 | 96.71 | 4.71 | 101.42 | <=2 |

Table 9 depicts symptom information for the PASC patients enrolled in the study.

| Symptoms | Symptoms details | P01 | P02 | P04 | P05 | P06 |
|---|---|---|---|---|---|---|
| Abdominal pain | Abdominal pain before T3 | | Yes | | Yes | |
| | Abdominal pain chart | No | No | Unknown | Yes | Unknown |
| | Abdominal pain COVID related | | Yes | | Yes | |
| | Abdominal pain interview | No | Yes | Unknown | Unknown | No |
| | Abdominal pain since onset COVID | | Yes | | Yes | |
| | Abdominal pain symptoms resolved | | True | | True | |
| | Abdominal pain started days since enrollment | | −8.5 | | −3.75 | |
| | Abdominal pain resolved days since enrollment | | 7.5 | | −1.75 | |
| Cough | Cough before T3 | Yes | Yes | Yes | Yes | Yes |
| | Cough chart | Yes | Yes | Yes | Yes | Yes |
| | Cough COVID related | Yes | Yes | Yes | Yes | Yes |
| | Cough interview | Yes | Yes | Unknown | Unknown | No |
| | Cough since onset COVID | Yes | Yes | Yes | Yes | Yes |
| | Cough symptoms resolved | True | True | True | True | False |
| | Cough started days since enrollment | −1.71 | −8.5 | −4.54 | −7.75 | −8.71 |
| | Cough resolved days since enrollment | 70.29 | 17.5 | −4.54 | −1.75 | |
| Diarrhea | Diarrhea before T3 | | Yes | | | |
| | Diarrhea chart | No | No | Unknown | No | No |
| | Diarrhea COVID related | | Yes | | | |
| | Diarrhea interview | No | Yes | Unknown | Unknown | No |
| | Diarrhea since onset COVID | | Yes | | | |
| | Diarrhea symptoms resolved | | True | | | |
| | Diarrhea started days since enrollment | | −8.5 | | | |
| | Diarrhea resolved days since enrollment | | 7.5 | | | |
| Dizziness | Dizziness before T3 | | | | Yes | |
| | Dizziness chart | Unknown | Unknown | Unknown | Yes | Unknown |
| | Dizziness COVID related | | | | Yes | |
| | Dizziness interview | Unknown | Unknown | Unknown | Unknown | Unknown |
| | Dizziness since onset COVID | | | | Yes | |
| | Dizziness symptoms resolved | | | | True | |
| | Dizziness started days since enrollment | | | | 19.25 | |
| | Dizziness resolved days since enrollment | | | | 22.25 | |
| Dyspnea | Shortness of breath before T3 | Yes | Yes | Yes | Yes | Yes |
| | Shortness of breath chart | Yes | Yes | Yes | Yes | Yes |
| | Shortness of breath COVID related | Yes | Yes | Yes | Yes | Yes |
| | Shortness of breath interview | Yes | Yes | Unknown | Unknown | Yes |
| | Shortness of breath since onset COVID | Yes | Yes | Yes | Yes | Yes |
| | Shortness of breath symptoms resolved | True | True | True | | True |
| | Shortness of breath started days since enrollment | −1.71 | −8.5 | −3.54 | −2.75 | −8.71 |
| | Shortness of breath resolved days since enrollment | 28.29 | 22.5 | 8.46 | | −263.71 |
| Fatigue | Fatigue before T3 | Yes | Yes | | | Yes |
| | Fatigue chart | Yes | No | Unknown | Unknown | Yes |
| | Fatigue COVID related | Yes | Yes | | | Yes |
| | Fatigue interview | No | Yes | Unknown | Unknown | Yes |
| | Fatigue since onset COVID | Yes | Yes | | | Yes |
| | Fatigue symptoms resolved | True | True | | | False |
| | Fatigue started days since enrollment | −1.71 | | | | −8.71 |
| | Fatigue resolved days since enrollment | 23.29 | 12.5 | | | |
| Fever | Fever or chills before T3 | | Yes | Yes | Yes | Yes |
| | Fever or chills chart | Unknown | Yes | Yes | Yes | Yes |
| | Fever or chills COVID related | | Yes | Yes | Yes | Yes |
| | Fever or chills interview | Unknown | No | Unknown | Unknown | No |
| | Fever or chills since onset COVID | | Yes | Yes | Yes | Yes |
| | Fever or chills symptoms resolved | | True | True | True | True |
| | Fever or chills started days since enrollment | | −8.5 | −3.54 | −3.75 | −10.71 |
| | Fever or chills resolved days since enrollment | | −5.5 | 2.46 | 22.25 | −0.71 |
| Hair loss | Hair loss before T3 | | | | | Yes |
| | Hair loss chart | Unknown | Unknown | Unknown | Unknown | No |
| | Hair loss COVID related | | | | | Yes |
| | Hair loss interview | Unknown | Unknown | Unknown | Unknown | Yes |
| | Hair loss since onset COVID | | | | | Yes |
| | Hair loss symptoms resolved | | | | | True |
| | Hair loss started days since enrollment | | | | | 42.29 |
| | Hair loss resolved days since enrollment | | | | | 116.29 |
| Headache | Headache before T3 | | | | Yes | |
| | Headache chart | Unknown | Unknown | Unknown | Yes | No |
| | Headache COVID related | | | | Yes | |
| | Headache interview | Unknown | Unknown | Unknown | Unknown | Unknown |
| | Headache since onset COVID | | | | Yes | |
| | Headache symptoms resolved | | | | True | |
| | Headache started days since enrollment | | | | 19.25 | |
| | Headache resolved days since enrollment | | | | 36.25 | |
| Loss of smell | Loss of smell before T3 | | Yes | | | |
| | Loss of smell chart | Unknown | Yes | Unknown | Unknown | Unknown |
| | Loss of smell COVID related | | Yes | | | |
| | Loss of smell interview | No | Yes | Unknown | Unknown | No |
| | Loss of smell since onset COVID | | Yes | | | |
| | Loss of smell symptoms resolved | | True | | | |
| | Loss of smell started days since enrollment | | −8.5 | | | |
| | Loss of smell resolved days since enrollment | | 22.5 | | | |
| Loss of taste | Loss of taste before T3 | Yes | Yes | | | |
| | Loss of taste chart | Yes | Yes | Unknown | Unknown | Unknown |
| | Loss of taste COVID related | Yes | Yes | | | |
| | Loss of taste interview | Yes | Yes | Unknown | Unknown | No |
| | Loss of taste since onset COVID | Yes | Yes | | | |
| | Loss of taste symptoms resolved | False | True | | | |
| | Loss of taste started days since enrollment | −1.71 | −8.5 | | | |
| | Loss of taste resolved days since enrollment | | 22.5 | | | |
| Memory problems | Memory problems before T3 | | | | | Yes |
| | Memory problems chart | Unknown | Unknown | Unknown | Unknown | Unknown |
| | Memory problems COVID related | | | | | Yes |
| | Memory problems interview | Unknown | Unknown | Unknown | Unknown | Yes |
| | Memory problems since onset COVID | | | | | Yes |
| | Memory problems symptoms resolved | | | | | False |
| | Memory problems started days since enrollment | | | | | 12.29 |
| | Memory problems resolved days since enrollment | | | | | |
| Muscle body aches | Muscle body aches before T3 | | Yes | | Yes | |
| | Muscle body aches chart | Unknown | Yes | Unknown | Yes | Unknown |
| | Muscle body aches COVID related | | Yes | | Yes | |
| | Muscle body aches interview | Unknown | Yes | Unknown | Unknown | Unknown |
| | Muscle body aches since onset COVID | | Yes | | Yes | |
| | Muscle body aches symptoms resolved | | TRUE | | TRUE | |
| | Muscle body aches started days since enrollment | | −8.5 | | −7.75 | |
| | Muscle body aches resolved days since enrollment | | 3.5 | | −1.75 | |
| Nausea | Nausea before T3 | | Yes | | Yes | |
| | Nausea chart | No | No | Unknown | Yes | No |
| | Nausea COVID related | | Yes | | Yes | |
| | Nausea interview | No | Yes | Unknown | Unknown | No |
| | Nausea since onset COVID | | Yes | | Yes | |
| | Nausea symptoms resolved | | True | | True | |
| | Nausea started days since enrollment | | −8.5 | | −7.75 | |
| | Nausea resolved days since enrollment | | 7.5 | | −1.75 | |
| Persistent chest pain | Persistent chest pain | | | | Yes | |
| | Persistent chest pain chart | Unknown | Unknown | Unknown | Yes | Unknown |
| | Persistent chest pain COVID related | | | | Yes | |
| | Persistent chest pain interview | Unknown | Unknown | Unknown | Unknown | Unknown |
| | Persistent chest pain since onset COVID | | | | Yes | |
| | Persistent chest pain symptoms resolved | | | | | |
| | Persistent chest pain started days since enrollment | | | | −7.75 | |
| | Persistent chest pain resolved days since enrollment | | | | | |
| Sputum | Sputum before T3 | | Yes | | | |
| | Sputum chart | Unknown | Yes | Unknown | Unknown | Unknown |
| | Sputum COVID related | | Yes | | | |
| | Sputum interview | No | Yes | Unknown | Unknown | No |
| | Sputum resolved days since enrollment | | 12.5 | | | |
| | Sputum since onset COVID | | Yes | | | |
| | Sputum started days since enrollment | | −8.5 | | | |
| | Sputum symptoms resolved | | True | | | |
| Other | Other before T3 | Yes | | Yes | | Yes |
| | Other chart | Yes | | Yes | | Yes |
| | Other COVID related | Yes | | Yes | | Yes |
| | Other interview | No | | Unknown | | Yes |
| | Other name | AKI | | Seizures | | Loss of |
| | | | | | | appetite |
| | Other resolved days since enrollment | 141.29 | | −2.54 | | |
| | Other since onset COVID | Yes | | Yes | | Yes |
| | Other started days since enrollment | −2.71 | | −3.54 | | −10.71 |
| | Other symptoms resolved | True | | True | | False |

Table 10 depicts List of MB designs.

| Gene | | | | |
|---|---|---|---|---|
| name | Gene ID | Location | MB | Target sequence |
| N2 | NC_ | 29190-29210 | 5′-+CTG +AAG /Cy3/ +CGC | AATTTGCCCCCAGCGCTTO |
| | 045512.2 | | +TGG +GGG +CAA ATT CCC | AG (SEQ ID NO: 16) |
| | | | CCA GCG/3BHQ_2/-3′ | |
| | | | (SEQ ID NO: 8) | |
| N4 | NC_ | 28699-28719 | 5′-+CGG +GTG /ICy3/+CCA | AAAAGATCACATTGGCACCC |
| | 045512.2 | | +ATG +TGA +TCT TTT GAG | G (SEQ ID NO: 17) |
| | | | ATC ACA TTG G/3BHQ_2/-3′ | |
| | | | (SEQ ID NO: 9) | |
| N7 | NC_ | 28531-28551 | 5′-+CGT +CTG /Cy3/+GTA | CTACCGAAGAGCTACCAGA |
| | 045512.2 | | +GCT +CTT +CGG TAG CGA | CG (SEQ ID NO: 18) |
| | | | AGA GOT AC/3BHQ_2/-3′ | |
| | | | (SEQ ID NO: 10) | |
| ΔF157 | B.1.617.2 | 22011-22028 | 5′-[Cy3][+C]CT [+C]AC | AAAGTTGGATGGAAAGTG |
| | (OX014251.1) | | [+T]T[+T] C[+C]A T[+C]C | (SEQ ID NO: 19) |
| | | | [+A]AC TTT TGG ATG GAA | |
| | | | A[BHQ2]-3′ | |
| | | | (SEQ ID NO: 11) | |
| L452R | B.1.617.2 | 22892-22917 | 5′-[Cy3][+C]T[+A] T[+A]C | GGTGGTAATTATAATTACCG |
| | (OX014251.1) | | [+C]G[+G] T[+A]A [+T]TA | GTATAG (SEQ ID NO: 20) |
| | | | [+T]AA TTA CCA CCA ATT | |
| | | | ATA ATT ACC G[BHQ2]-3′ | |
| | | | (SEQ ID NO: 12) | |
| ΔH69 | BA.1 | 21682-21703 | 5′-+CCA +GAT /Cy3/+ATA | CTTGGTTCCATGCTATATCT |
| | (OR624070.1) | | +GCA +TGG +AAC CAA GTT | GG (SEQ ID NO: 21) |
| | | | CCA TGC TAT /3BHQ_2/-3′ | |
| | | | (SEQ ID NO: 13) | |
| Influenza | OR_637833.1 | 616-637 | 5′-+GTC +CAT /CY3/+TTT | TTCTGGAGGGGTGAAAATG |
| A | | | +CAC +CCC +TCC AGA AGA | GAC (SEQ ID NO: 22) |
| (H1N1, | | | GGG GTG AAA /3BHQ_21-3′ | |
| NP | | | (SEQ ID NO: 14) | |
| gene) | | | | |
| RSV | OQ_848527.1 | 5944-5966 | 5′-+CTG +GCT /Cy3/+CGA | CAAGCAACAAACAATCGAGC |
| | | | +TTG +TTT +GTT GCT TGA | CAG (SEQ ID NO: 23) |
| | | | ACA AAC AAT CG /3BHQ_2/- | |
| | | | 3′ | |
| | | | (SEQ ID NO: 15) | |

1. A method of detecting a viral antigen and a viral nucleic acid in a subject, comprising:
obtaining a biological sample from the subject;
capturing a virion from the biological sample; or
capturing a membranous particle from the biological sample; and
measuring the viral antigen and the viral nucleic acid level in the biological sample;
wherein the virion or membranous particle is immobilized on a biochip.

2. The method of claim 1, wherein the biochip comprises a plasmonic surface.

3. The method of claim 1, wherein the viral nucleic acid comprises RNA or DNA.

4. The method of claim 1, wherein the viral antigen comprises a viral protein.

5. The method of claim 1, wherein the viral antigen or viral nucleic acid is detected when the viral antigen and the viral nucleic acid levels are increased in comparison to a control sample.

6. The method of claim 1, wherein the virion comprises a coronavirus virion, an influenza virus virion, or a respiratory syncytial virus (RSV) virion.

7. The method of claim 1, wherein the membranous particle comprises a virion-infected host-derived extracellular vesicle.

8. The method of claim 1, wherein the biological sample is saliva.

9. The method of claim 1, wherein the biological sample is a nasopharyngeal swab.

10. The method of claim 1, wherein the biological sample is a plasma sample.

11. The method of claim 1, wherein the viral nucleic acid is measured using one or more probes complementary to a target nucleic acid sequence.

12. The method of claim 1, further comprising administering an antiviral agent if the viral antigen or the viral nucleic acid is detected.

13. A method of treating a viral infection in a subject, comprising:
obtaining a biological sample from the subject;
capturing a virion from the biological sample; or
capturing a membranous particle from the biological sample;
measuring a viral antigen or a viral nucleic acid level in the biological sample;
wherein the virion or membranous particle is immobilized on a biochip; and
administering an antiviral agent if a viral infection is detected.

14. The method of claim 13, wherein the biochip comprises of a plasmonic surface.

15. The method of claim 13, wherein the viral infection is detected when the viral antigen or nucleic acid level is increased in comparison to a control sample.

16. A method for high throughput multiplexing for simultaneous detection of a viral antigen and a viral nucleic acid, the method comprising the steps of:
immobilizing a virion and/or a membranous particle on a plasmonic surface;
detecting the viral antigen and the viral nucleic acid simultaneously derived from the virion and/or membranous particle;
wherein the detection method comprises fluorescence, in situ hybridization, enzyme linked immunosorbent assay, flow cytometry, or microscopy.

17. The method of claim 16, wherein the viral antigen and the viral nucleic acid are detected with a sensitivity of at least 80%.

18. The method of claim 16, wherein the viral antigen and the viral nucleic acid are detected with a specificity of at least 95%.
